(19) United States
(12) Patent Application Publication
(10) Pub. No.: US 2026/0324653 A1
(43) Pub. Date: Oct. 22, 2026

(54) INSECTICIDAL PROTEIN AND USE THEREOF

(71) Applicant: Beijing Dabeinong Biotechnology Co., Ltd., Beijing (CN)

(72) Inventors: Liangwei Zhang, Beijing (CN); Ming Zhang, Beijing (CN); Xiangting Xie, Beijing (CN); Yuhan Gao, Beijing (CN); Jie Pang, Beijing (CN); Qinyang Wang, Beijing (CN)

(73) Assignee: Beijing Dabeinong Biotechnology Co., Ltd, Beijing (CN)

(21) Appl. No.: 19/484,320

(22) PCT Filed: Dec. 23, 2024
(86) PCT No.: PCT/CN2024/141426

§ 371 Date: Nov. 13, 2025

(30) Foreign Application Priority Data
Mar. 27, 2024 (CN) 202410361677.1

(51) Int. Cl. A01N 63/50; A01P 7/04; C07K 14/325

(52) U.S. Cl. CPC A01N 63/50; A01P 7/04; C07K 14/325

(57) ABSTRACT

An insecticidal protein, a nucleic acid molecule encoding the protein, and a method and the use thereof for controlling Lepidoptera pests. The insecticidal protein contains an amino acid sequences as set forth in SEQ ID NO: 7, SEQ ID NO: 8, SEQ ID NO: 10, SEQ ID NO: 11 or SEQ ID NO: 33. The modified Cry1Da1 protein has a better insecticidal effect, with significantly greater insecticidal activity compared to the patented protein BD1-002.

### FIELD OF THE INVENTION

The present invention relates to an insecticidal protein, a nucleic acid molecule encoding the protein, and a method and use thereof for controlling Lepidoptera pests.

### BACKGROUND

Currently, agricultural production faces biotic stress (such as diseases and pests) and abiotic stress (such as drought, cold, and salt damage), which weaken crop growth and reduce yield, posing a serious threat to global food security. Among these, pests are one of the major biotic stress factors affecting agricultural and forestry productivity. As the environmental problems caused by the use of chemical pesticides for pest control become increasingly severe, the use of biological insecticides is gradually aroused people's attention.

Bacillus thuringiensis (abbreviated as Bt) is a gram-positive bacterium widely distributed in nature. The most distinct feature that distinguished Bt bacteria from other Bacillus species is the formation of crystal proteins along with spore formation during the late stage of growth, which are generally referred to as parasporal crystals. Bt strain is considered as an insect pathogen, and its pathogenicity mainly or completely depends on the parasporal crystal protein. In recent years, a large number of literature have reported that various Bt proteins have insecticidal activity against insects, such as Lepidoptera, Coleoptera, Diptera, Hymenoptera, and Homoptera insects. The commercial cultivation of Bt transgenic insect-resistant crops has become one of the major drivers for significantly improving agricultural productivity. In recent years, the industrialization scale of transgenic insect-resistant crops is continuously expanding, effectively controlling the occurrence and damage of target pests, reducing the application of chemical insecticides, and providing important guarantee for food and ecological security.

Long-term and large-scale cultivation of Bt transgenic crops may lead to the development of Bt resistance in pests, which is a key factor for affecting the sustainable application of transgenic insect-resistant crops. In order to delay the evolution of pest resistance to Bt crops, the “High Dose/Refuge” strategy has been widely used globally. The theoretical basis of the “High Dose/Refuge” includes two parts: “High Dose” and “Refuge”. The “Refuge” refers to non-transgenic plant hosts pests near Bt crops that provide survival space for sensitive individuals of target. The “High Dose” refers to the high dose of insecticidal protein expressed by transgenic insect-resistant crops, which theoretically can kill 100% of sensitive individuals and 95% of sensitive/resistant heterozygous individuals in the target pest population. In this regard, the requirements of various countries are relatively consistent: defining the concentration that is 25 times the 99% lethal dose for sensitive target pests as the standard, and the concentration higher than this concentration as high dose. Nevertheless, due to the limitations of existing biotechnology and plant carrying capacity, the dose of Bt protein expressed in transgenic crops cannot be infinitely increased. Moreover, lower Bt expression levels are beneficial for reducing metabolic consumption in transgenic plants and improving agronomic traits. Therefore, improving the insecticidal effect of Bt protein in transgenic crops is crucial for the sustainable application of insect-resistant transgenic crops.

Cry1Da1 protein, a parasporal crystal protein of Bacillus thuringiensis, is a Lepidoptera-specific Bt insecticidal crystal protein with a new mechanism. Its intestinal receptor in Lepidoptera is different from those of other Cry proteins (Reference 1), and it has good resistance to Lepidoptera insects, especially the pests of Spodoptera litura family.

Nevertheless, the insecticidal activity of the Cry1Da1 protein disclosed in the prior art still cannot meet the increasing demands for pest control in agricultural production. Therefore, there is an urgent need for the Cry1Da1 protein with better insecticidal effects.

### SUMMARY OF THE INVENTION

In view of this, the purpose of the present invention is to provide a Cry1Da1 protein with better insecticidal effects.

In a first aspect, the present invention provides an insecticidal protein comprising the amino acid sequence as shown in SEQ ID NO: 7, SEQ ID NO: 8, SEQ ID NO: 10, SEQ ID NO: 11, or SEQ ID NO: 33.

In a second aspect, the present invention provides a nucleic acid molecule encoding the insecticidal protein of the present invention.

Preferably, the nucleotide sequences encoding the amino acid sequences as shown in SEQ ID NO: 7, SEQ ID NO: 8, SEQ ID NO: 10, SEQ ID NO: 11, or SEQ ID NO: 33, are shown in SEQ ID NO: 18, SEQ ID NO: 19, SEQ ID NO: 21, SEQ ID NO: 22, or SEQ ID NO: 34, respectively.

In a third aspect, the present invention provides a recombinant expression vector comprising the nucleic acid molecule of the present invention.

In a fourth aspect, the present invention provides an insecticidal composition comprising the insecticidal protein of the present invention.

In a fifth aspect, the present invention provides a method for controlling Lepidoptera pests, comprising contacting Lepidoptera pests with the insecticidal protein or insecticidal composition of the present invention.

In a sixth aspect, the present invention provides a method for controlling Lepidoptera pests, comprising introducing the nucleic acid molecule or recombinant expression vector of the present invention into a plant, and making Lepidoptera pests feed on the plant.

In a seventh aspect, the present invention provides use of the insecticidal protein, nucleic acid molecule, recombinant expression vector, or insecticidal composition for controlling Lepidoptera pests.

The beneficial effects of the present invention are as follows:

The modified Cry1Da1 protein obtained by the present invention significantly increases the mortality rate of Lepidoptera pests when fed to them. Meanwhile, transgenic plants expressing the modified Cry1Da1 protein of the present application also exhibit superior insect-resistant effects. After inoculated with Lepidoptera pests, the transgenic plants have a lower leaf damage rate and a higher mortality rate of Lepidoptera pests. Therefore, the modified Cry1Da1 protein obtained by the present invention has a better insect-resistant activity, and its effect is significantly better than the patented protein BD1-002.

### BRIEF DESCRIPTION OF THE DRAWINGS

FIG. 1 shows the flow diagram of constructing the recombinant cloning vector DBN002A containing the nucleotide sequence of the Cry1Da1-related protein BD1-002 according to the present invention.

FIG. 2 shows the flow diagram of constructing the soybean recombinant expression vector DBN002A-B containing the nucleotide sequence of the Cry1Da1-related protein BD1-002 according to the present invention.

FIG. 3 shows the flow diagram of constructing the corn recombinant expression vector DBN002A-C containing the nucleotide sequence of the Cry1Da1-related protein BD1-002 according to the present invention.

### DETAILED DESCRIPTION OF THE INVENTION

It should be noted that, unless otherwise defined, the technical or scientific terms used in the present application should have the common meanings understood by those skilled in the art.

The experimental methods in the following Examples, unless otherwise specified, are all conventional methods. The medicinal raw materials, reagents and materials, and the like used in the following examples are all commercially available products unless otherwise specified.

In a first aspect, the present invention provides an insecticidal protein comprising the amino acid sequence as shown in SEQ ID NO: 7, SEQ ID NO: 8, SEQ ID NO: 10, SEQ ID NO: 11, or SEQ ID NO: 33.

Those skilled in the art will easily recognize that advances in the field of molecular biology, such as site-specific mutagenesis and random mutagenesis, polymerase chain reaction methods, and protein engineering techniques, provide a wide range of appropriate tools and operational steps for modifying or engineering the amino acid sequences and potential gene sequences of proteins of agricultural interest.

The genes and proteins in the present invention not only include specific exemplary sequences, but also include parts and/or fragments (including internal and/or terminal deletions compared to full-length proteins), variants, mutants, substitutions (proteins with substituted amino acids), chimeras, and fusion proteins that retain the insecticidal activity characteristics of the specific exemplary proteins. The term “variant” or “variation” refers to the nucleotide sequence encoding the same protein or an equivalent protein with insecticidal activity. The term “equivalent protein” refers to a protein that has the same or essentially the same biological activity against Lepidoptera pests as the protein in the present invention.

In the present invention, the modified Cry1Da1 protein includes but is not limited to amino acid sequences that have a certain homology with the amino acid sequence as shown in SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 33. These sequences typically have a similarity/identity of greater than 60%, preferably greater than 75%, more preferably greater than 90%, even more preferably greater than 95%, and can have a similarity/identity of greater than 99% with the sequences of the present invention. The preferred nucleotides and proteins of the present invention can also be defined based on more specific ranges of identity and/or similarity. For example, they can have an identity and/or similarity of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the exemplary sequences of the present invention.

In a second aspect, the present invention provides a nucleic acid molecule encoding the insecticidal protein of the present invention. Preferably, the nucleotide sequences encoding the amino acid sequences as shown in SEQ ID NO: 7, SEQ ID NO: 8, SEQ ID NO: 10, SEQ ID NO: 11, or SEQ ID NO: 33, are shown in SEQ ID NO: 18, SEQ ID NO: 19, SEQ ID NO: 21, SEQ ID NO: 22, or SEQ ID NO: 34, respectively.

The nucleic acid molecule or fragment thereof in the present invention hybridizes with the modified Cry1Da1 gene under stringent conditions. Any conventional nucleic acid hybridization or amplification method can be used to identify the presence of the modified Cry1Da1 gene of the present invention. The nucleic acid molecules or the fragments thereof can specifically hybridize with other nucleic acid molecules under certain circumstances. In the present invention, if two nucleic acid molecules can form an antiparallel double-stranded nucleic acid structure, it can be said that these two nucleic acid molecules can specifically hybridize with each other. If two nucleic acid molecules exhibit complete complementarity, then one of the nucleic acid molecules is referred to as the “complement” of the other nucleic acid molecule. In the present invention, when each nucleotide of a nucleic acid molecule is complementary to the corresponding nucleotide of another nucleic acid molecule, then the two nucleic acid molecules are said to exhibit “complete complementarity”. If two nucleic acid molecules can hybridize with each other with sufficient stability to allow them to anneal and bind to each other under at least conventional “low-stringency” conditions, then these two nucleic acid molecules are said to be “minimally complementary”. Similarly, if two nucleic acid molecules can hybridize with each other with sufficient stability to allow them to anneal and bind to each other under conventional “high-stringency” conditions, then these two nucleic acid molecules are said to have “complementarity”. Deviation from complete complementarity is allowed as long as it does not completely prevent two molecules from forming a double-stranded structure. For a nucleic acid molecule to function as a primer or probe, it is only necessary to ensure that the nucleic acid molecule has sufficient complementarity in sequence to form a stable double-stranded structure under the specific solvent and salt concentration used.

In the present invention, an essentially homologous sequence is a nucleic acid molecule that can specifically hybridize with the complementary strand of another matching nucleic acid molecule under high-stringency conditions. Suitable stringent conditions for promoting DNA hybridization, such as treatment with 6.0×sodium chloride/sodium citrate (SSC) at approximately 45° C., followed by washing with 2.0×SSC at 50° C., are well known to those skilled in the art. For example, the salt concentration in the washing step can range from about 2.0×SSC at 50° C. for low-stringency conditions to about 0.2×SSC at 50° C. for high-stringency conditions. In addition, the temperature condition in the washing step can range from room temperature (about 22° C.) for low-stringency conditions to about 65° C. for high-stringency conditions. The temperature condition and salt concentration can be changed, or one can remain unchanged while the other is changed. Preferably, the stringent condition in the present invention can comprise specific hybridization at 65° C. in a 6×SSC, 0.5% SDS solution with SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 34, followed by respectively washing the membrane once with 2×SSC, 0.1% SDS, and 1×SSC, 0.1% SDS.

Therefore, the sequences that have insecticidal activity and hybridize with SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 34 of the present invention under stringent conditions are included in the present invention. These sequences have at least approximately 40%-50% homology, approximately 60%, 65% or 70% homology, or even at least approximately 75%, 80%, 85%, 90%, 91%, 92%, 93%, 94%, 95%, 96%, 97%, 98%, 99% or greater sequence homology with the sequences of the present invention.

Due to the abundancy of genetic codon, multiple different DNA sequences can encode the same amino acid sequence. The generation of alternative DNA sequences encoding the same protein or essentially the same protein is within the technical capabilities of those skilled in the art. These different DNA sequences are included within the scope of the present invention. The term “essentially the same” sequence refers to the sequence with amino acid substitutions, deletions, additions, or insertions that do not substantially affect insecticidal activity, also including the fragments that retain insecticidal activity.

In a third aspect, the present invention provides a recombinant expression vector comprising the nucleic acid molecule of the present invention.

The term “transgenic” in the present invention refers to any cell, cell line, callus, tissue, plant part, or plant whose genome has been altered by the presence of a heterologous nucleic acid (such as a recombinant DNA construct). The term “transgenic” in the present invention includes those initial transgenic events as well as those produced from the initial transgenic events through sexual hybridization or asexual reproduction, and does not cover genomic (chromosomal or extrachromosomal) changes produced through conventional plant breeding methods or through naturally occurring events, such as random cross-fertilization, non-recombinant viral infection, non-recombinant bacterial transformation, non-recombinant transposition, or spontaneous mutation.

In a fourth aspect, the present invention provides an insecticidal composition comprising the insecticidal protein of the present invention.

The term “insecticidal” or “insect-resistant” in the present invention refers to being toxic to crop pests, thereby achieving “control” and/or “prevention” of crop pests. Preferably, the term “insecticidal” or “insect-resistant” refers to killing crop pests. More specifically, the target insect is a Lepidoptera pest.

In a fifth aspect, the present invention provides a method for controlling Lepidoptera pests, comprising contacting Lepidoptera pests with the insecticidal protein or insecticidal composition of the present invention.

In a sixth aspect, the present invention provides a method for controlling Lepidoptera pests, comprising introducing the nucleic acid molecule or recombinant expression vector of the present invention into a plant, and making Lepidoptera pests feed on said plant.

The term “contact” in the present invention refers to touching, staying, and/or feeding, specifically touching, staying, and/or feeding of insects and/or pests on plants, plant organs, plant tissues, or plant cells. The plants, plant organs, plant tissues, or plant cells may either express insecticidal proteins in vivo, or the plants, plant organs, plant tissues, or plant cells have insecticidal proteins and/or insecticidal protein-producing microorganisms on their surfaces.

The terms “control” and/or “prevention” in the present invention refer to the inhibition of growth and/or death of Lepidoptera pests after they come into contact with the Cry1Da1 protein and the modified proteins thereof. Furthermore, Lepidoptera pests come into contact with the Cry1Da1 protein by feeding on plant tissues, which results in the inhibition and/or death of all or part of the Lepidoptera pests. Inhibition refers to sublethal effects, which are not lethal but can cause certain effects on growth, development, behavior, physiology, biochemistry, tissue, and so on, such as slowed and/or halted growth and development. Meanwhile, the plants should be morphologically normal, and can be cultured by conventional methods for consumption and/or production of products. In addition, plants and/or plant seeds that contain the nucleotide sequences encoding the Cry1Da1 protein and control Lepidoptera pests have reduced plant damage compared to non-transgenic wild-type plants under conditions of artificial inoculation of Lepidoptera pests and/or damage caused by naturally occurring Lepidoptera pests. Specific manifestations include, but are not limited to, improved leaf resistance, and/or increased grain weight, and/or increased yield. The “control” and/or “prevention” effect of the Cry1Da1 protein on Lepidoptera pests can exist independently. Specifically, if any tissue of a transgenic plant (containing the nucleotide sequence encoding the Cry1Da1 protein) simultaneously and/or asynchronously contains and/or produces the Cry1Da1 protein and/or another substance that can control Lepidoptera pests, then the presence of said another substance cannot cause the “control” and/or “prevention” effect to be fully and/or partially achieved by said another substance, independent of the Cry1Da1 protein. Typically, in the field, the feeding process of Lepidoptera pests on plant tissues is brief and difficult to observe with naked eye. Therefore, under conditions of artificial inoculation of Lepidoptera pests and/or damage caused by naturally occurring Lepidoptera pests, for example, if there are dead Lepidoptera pests on any tissue of a transgenic plant (containing the nucleotide sequence encoding the Cry1Da1 protein), and/or there are Lepidoptera pests with inhibited growth staying on any tissue of a transgenic plant (containing the nucleotide sequence encoding the Cry1Da1 protein), and/or any tissue of a transgenic plant (containing the nucleotide sequence encoding the Cry1Da1 protein) has reduced plant damage compared to non-transgenic wild-type plants, then the method and/or use of the present invention is achieved; that is, the method and/or use of controlling Lepidoptera pests is achieved by contacting Lepidoptera pests with the Cry1Da1 protein.

In a preferred embodiment, the Lepidoptera pests are Spodoptera frugiperda, Helicoverpa armigera, or Argyrogramma agnata.

In a preferred embodiment, the plant is a monocotyledon or a dicotyledon; in a more preferred embodiment, the plant is corn or soybean.

The term “plant” in the present invention refers to any plant, including whole plants, plant cells, plant organs, plant protoplasts, plant cell tissue cultures from which plants can be regenerated, plant callus, and intact plant cells in plants or plant parts, wherein the plant parts are, for example, embryos, pollen, ovules, seeds, leaves, flowers, branches, fruits, stems, roots, root tips, anthers, and the like.

In a seventh aspect, the present invention provides use of the insecticidal protein, the nucleic acid molecule, the recombinant expression vector, or the insecticidal composition of the present invention for controlling Lepidoptera pests.

In a preferred embodiment, the Lepidoptera pests are Spodoptera frugiperda, Helicoverpa armigera, or Argyrogramma agnata.

The amino acid sequences and nucleotide sequences involved herein are shown in the following table:

| | | SEQ ID |
|---|---|---|
| Name | Sequence Information | NO |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 1 |
| 001 | FLSDWIVYNRFRRQLTISVLDIVAFFPNYDIRTYPIQTATQLTREVYLDLPFINEN | |
| | LSPAASYPTFSAAESAIIRSPHLVDFLNSFTIYTDSLARYAYWGGHLVNSFRTGT | |
| | TTNLIRSPLYGREGNTERPVTITASPSVPIFRTLSYITGLDNSNPVAGIEGVEFQNT | |
| | ISRSIYRKSGPIDSFSELPPQDASVSPAIGYSHRLCHATFLERISGPRIAGTVFSWT | |
| | HRSASPTNEVSPSRITQIPWVKAHTLASGASVIKGPGFTGGDILTRNSMGELGTL | |
| | RVTFTGRLPQSYYIRFRYASVANRSGTFRYSQPPSYGISFPKTMDAGEPLTSRSF | |
| | AHTTLFTPITFSRAQEEFDLYIQSGVYIDRIEFIPVTATFEAEYDLERAQKVVNAL | |
| | FTSTNQLGLKTDVTDYHIDQVSNLVACLSDEFCLDEKRELSEKVKHAKRLSDER | |
| | NLLQDPNFRGINRQPDRGWRGSTDITIQGGDDVFKENYVTLPGTFDECYPTYLY | |
| | QKIDESKLKAYTRYQLRGYIEDSQDLEIYLIRYNAKHEIVNVPGTGSLWPLSVEN | |
| | QIGPCGEPNRCAPHLEWNPDLHCSCRDGEKCAHHSHHFSLDIDVGCTDLNEDL | |
| | GVWVIFKIKTQDGHARLGNLEFLEEKPLLGEALARVKRAEKKWRDKRETLQLE | |
| | TTIVYKEAKESVDALFVNSQYDRLQADTNIAMIHAADKRVHRIREAYLPELSVI | |
| | PGVNAAIFEELEERIFTAFSLYDARNIIKNGDFNNGLLCWNVKGHVEVEEQNNH | |
| | RSVLVIPEWEAEVSQEVRVCPGRGYILRVTAYKEGYGEGCVTIHEIENNTDELK | |
| | FNNCVEEEVYPNNTVTCINYTATQEEYEGTYTSRNRGYDEAYGNNPSVPADYA | |
| | SVYEEKSYTDRRRENPCESNRGYGDYTPLPAGYVTKELEYFPETDKVWIEIGET | |
| | EGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 2 |
| 002 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYPTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKVVNA | |
| | LFTSTNQLGLKTDVTDYHIDQVSNLVACLSDEFCLDEKRELSEKVKHAKRLSDE | |
| | RNLLQDPNFRGINRQPDRGWRGSTDITIQGGDDVFKENYVTLPGTFDECYPTYL | |
| | YQKIDESKLKAYTRYQLRGYIEDSQDLEIYLIRYNAKHEIVNVPGTGSLWPLSVE | |
| | NQIGPCGEPNRCAPHLEWNPDLHCSCRDGEKCAHHSHHFSLDIDVGCTDLNED | |
| | LGVWVIFKIKTQDGHARLGNLEFLEEKPLLGEALARVKRAEKKWRDKRETLQL | |
| | ETTIVYKEAKESVDALFVNSQYDRLQADTNIAMIHAADKRVHRIREAYLPELSV | |
| | IPGVNAAIFEELEERIFTAFSLYDARNIIKNGDFNNGLLCWNVKGHVEVEEQNN | |
| | HRSVLVIPEWEAEVSQEVRVCPGRGYILRVTAYKEGYGEGCVTIHEIENNTDEL | |
| | KFNNCVEEEVYPNNTVTCINYTATQEEYEGTYTSRNRGYDEAYGNNPSVPADY | |
| | ASVYEEKSYTDRRRENPCESNRGYGDYTPLPAGYVTKELEYFPETDKVWIEIGE | |
| | TEGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 3 |
| 011 | FLSDWIVYNRFRRQLTISVLDIVAFFPNYDIRTYPIQTATQLTREVYLDLPFINEN | |
| | LSPAALYPTFSAAESAIIRSPHLVDFLNSFTIYTDSLARSAYWGGHLVNSFRTGTT | |
| | TNLIRSPLYGREGNTERPVTITASPSVPIFRTLSYPTGLDNSNPVAGIEGVEFQNTI | |
| | SRSIYRKSGPIDSFSELPPQDASVSPAIGYSHRLCHATFLERISGPRIAGTVFSWTH | |
| | RSASPTNEVSPSRITQIPWVKAHTLASGASVIKGPGFTGGDILTRNSMGELGTLR | |
| | VTFTGRLPQSYYIRFRYASVANRSGTFRYSQPPSYGISFPKTMDAGEPLTSRSFA | |
| | HTTLFTPITFSRAQEEFDLYIQSGVYIDRIEFIPVTATFEAEYDLERAQKVVNALF | |
| | TSTNQLGLKTDVTDYHIDQVSNLVACLSDEFCLDEKRELSEKVKHAKRLSDER | |
| | NLLQDPNFRGINRQPDRGWRGSTDITIQGGDDVFKENYVTLPGTFDECYPTYLY | |
| | QKIDESKLKAYTRYQLRGYIEDSQDLEIYLIRYNAKHEIVNVPGTGSLWPLSVEN | |
| | QIGPCGEPNRCAPHLEWNPDLHCSCRDGEKCAHHSHHFSLDIDVGCTDLNEDL | |
| | GVWVIFKIKTQDGHARLGNLEFLEEKPLLGEALARVKRAEKKWRDKRETLQLE | |
| | TTIVYKEAKESVDALFVNSQYDRLQADTNIAMIHAADKRVHRIREAYLPELSVI | |
| | PGVNAAIFEELEERIFTAFSLYDARNIIKNGDFNNGLLCWNVKGHVEVEEQNNH | |
| | RSVLVIPEWEAEVSQEVRVCPGRGYILRVTAYKEGYGEGCVTIHEIENNTDELK | |
| | FNNCVEEEVYPNNTVTCINYTATQEEYEGTYTSRNRGYDEAYGNNPSVPADYA | |
| | SVYEEKSYTDRRRENPCESNRGYGDYTPLPAGYVTKELEYFPETDKVWIEIGET | |
| | EGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 4 |
| 013 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYFTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKVVNA | |
| | LFTSTNQLGLKTDVTDYHIDQVSNLVACLSDEFCLDEKRELSEKVKHAKRLSDE | |
| | RNLLQDPNFRGINRQPDRGWRGSTDITIQGGDDVFKENYVTLPGTFDECYPTYL | |
| | YQKIDESKLKAYTRYQLRGYIEDSQDLEIYLIRYNAKHEIVNVPGTGSLWPLSVE | |
| | NQIGPCGEPNRCAPHLEWNPDLHCSCRDGEKCAHHSHHFSLDIDVGCTDLNED | |
| | LGVWVIFKIKTQDGHARLGNLEFLEEKPLLGEALARVKRAEKKWRDKRETLQL | |
| | ETTIVYKEAKESVDALFVNSQYDRLQADTNIAMIHAADKRVHRIREAYLPELSV | |
| | IPGVNAAIFEELEERIFTAFSLYDARNIIKNGDFNNGLLCWNVKGHVEVEEQNN | |
| | HRSVLVIPEWEAEVSQEVRVCPGRGYILRVTAYKEGYGEGCVTIHEIENNTDEL | |
| | KFNNCVEEEVYPNNTVTCINYTATQEEYEGTYTSRNRGYDEAYGNNPSVPADY | |
| | ASVYEEKSYTDRRRENPCESNRGYGDYTPLPAGYVTKELEYFPETDKVWIEIGE | |
| | TEGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 5 |
| 014 | FLSDWIVYNRFRRQLTISVLDIVAFFPNYDIRTYPIQTATQLTREVYLDLPFINEN | |
| | LSPAALYPTFSAAESAIIRSPHLVDFLNSFTIYTDSLARSAYWGGHLVNSFRTGTT | |
| | TNLIRSPLYGREGNTERPVTITASPSVPIFRTLSYFTGLDNSNPVAGIEGVEFQNTI | |
| | SRSIYRKSGPIDSFSELPPQDASVSPAIGYSHRLCHATFLERISGPRIAGTVFSWTH | |
| | RSASPTNEVSPSRITQIPWVKAHTLASGASVIKGPGFTGGDILTRNSMGELGTLR | |
| | VTFTGRLPQSYYIRFRYASVANRSGTFRYSQPPSYGISFPKTMDAGEPLTSRSFA | |
| | HTTLFTPITFSRAQEEFDLYIQSGVYIDRIEFIPVTATFEAEYDLERAQKVVNALF | |
| | TSTNQLGLKTDVTDYHIDQVSNLVACLSDEFCLDEKRELSEKVKHAKRLSDER | |
| | NLLQDPNFRGINRQPDRGWRGSTDITIQGGDDVFKENYVTLPGTFDECYPTYLY | |
| | QKIDESKLKAYTRYQLRGYIEDSQDLEIYLIRYNAKHEIVNVPGTGSLWPLSVEN | |
| | QIGPCGEPNRCAPHLEWNPDLHCSCRDGEKCAHHSHHFSLDIDVGCTDLNEDL | |
| | GVWVIFKIKTQDGHARLGNLEFLEEKPLLGEALARVKRAEKKWRDKRETLQLE | |
| | TTIVYKEAKESVDALFVNSQYDRLQADTNIAMIHAADKRVHRIREAYLPELSVI | |
| | PGVNAAIFEELEERIFTAFSLYDARNIIKNGDFNNGLLCWNVKGHVEVEEQNNH | |
| | RSVLVIPEWEAEVSQEVRVCPGRGYILRVTAYKEGYGEGCVTIHEIENNTDELK | |
| | FNNCVEEEVYPNNTVTCINYTATQEEYEGTYTSRNRGYDEAYGNNPSVPADYA | |
| | SVYEEKSYTDRRRENPCESNRGYGDYTPLPAGYVTKELEYFPETDKVWIEIGET | |
| | EGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 6 |
| 015 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARTAYWGGHLVNSFRTGT | |
| | TTNLIRSPLYGREGNTERPVTITASPSVPIFRTLSYPTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKVVNA | |
| | LFTSTNQLGLKTDVTDYHIDQVSNLVACLSDEFCLDEKRELSEKVKHAKRLSDE | |
| | RNLLQDPNFRGINRQPDRGWRGSTDITIQGGDDVFKENYVTLPGTFDECYPTYL | |
| | YQKIDESKLKAYTRYQLRGYIEDSQDLEIYLIRYNAKHEIVNVPGTGSLWPLSVE | |
| | NQIGPCGEPNRCAPHLEWNPDLHCSCRDGEKCAHHSHHFSLDIDVGCTDLNED | |
| | LGVWVIFKIKTQDGHARLGNLEFLEEKPLLGEALARVKRAEKKWRDKRETLQL | |
| | ETTIVYKEAKESVDALFVNSQYDRLQADTNIAMIHAADKRVHRIREAYLPELSV | |
| | IPGVNAAIFEELEERIFTAFSLYDARNIIKNGDFNNGLLCWNVKGHVEVEEQNN | |
| | HRSVLVIPEWEAEVSQEVRVCPGRGYILRVTAYKEGYGEGCVTIHEIENNTDEL | |
| | KFNNCVEEEVYPNNTVTCINYTATQEEYEGTYTSRNRGYDEAYGNNPSVPADY | |
| | ASVYEEKSYTDRRRENPCESNRGYGDYTPLPAGYVTKELEYFPETDKVWIEIGE | |
| | TEGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 7 |
| 018 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYFTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKAVNA | |
| | LFTSTNQLGLKTNVTDYHIDQVSNLVTYLSDEFCLDEKRELSEKVKHAKRLSDE | |
| | RNLLQDPNFRGINRQPDRGWRGSTDITIQGGDDVFKENYVTLSGTFDECYPTYL | |
| | YQKIDESKLKAYTRYQLRGYIEDSQDLEIYLIRYNAKHETVNVPGTGSLWPLSV | |
| | ENQIGPCGEPNRCAPHLEWNPDLHCSCRDGEKCAHHSHHFSLDIDVGCTDLNE | |
| | DLGVWVIFKIKTQDGHARLGNLEFLEEKPLVGEALARVKRAEKKWRDKREKL | |
| | EWETNIVYKEAKESVDALFVNSQYDQLQADTNIAMIHAADKRVHSIREAYLPE | |
| | LSVIPGVNAAIFEELEGRIFTAFSLYDARNVIKNGDFNNGLSCWNVKGHVDVEE | |
| | QNNQRSVLVVPEWEAEVSQEVRVCPGRGYILRVTAYKEGYGEGCVTIHEIENN | |
| | TDELKFSNCVEEEIYPNNTVTCNDYTVNQEEYGGAYTSRNRGYNEAYGNNPSV | |
| | PADYASVYEEKSYTDGRRENPCEFNRGYRDYTPLPVGYVTKELEYFPETDKVW | |
| | IEIGETEGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 8 |
| 019 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYFTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATLEAEYNLERAQKAVNA | |
| | LFTSTNQLGLKTNVTDYHIDQVSNLVTYLSDEFCLDEKRELSEKVKHAKRLSDE | |
| | RNLLQDPNFRGINRQPDRGWRGSTDITIQGGDDVFKENYVTLSGTFDECYPTYL | |
| | YQKIDESKLKAYTRYQLRGYIEDSQDLEIYLIRYNAKHETVNVPGTGSLWPLSV | |
| | ENQIGPCGEPNRCAPHLEWNPDLHCSCRDGEKCAHHSHHFSLDIDVGCTDLNE | |
| | DLGVWVIFKIKTQDGHARLGNLEFLEEKPLVGEALARVKRAEKKWRDKREKL | |
| | EWETNIVYKEAKESVDALFVNSQYDQLQADTNIAMIHAADKRVHSIREAYLPE | |
| | LSVIPGVNAAIFEELEGRIFTAFSLYDARNVIKNGDFNNGLSCWNVKGHVDVEE | |
| | QNNQRSVLVVPEWEAEVSQEVRVCPGRGYILRVTAYKEGYGEGCVTIHEIENN | |
| | TDELKFSNCVEEEIYPNNTVTCNDYTVNQEEYGGAYTSRNRGYNEAYGNNPSV | |
| | PADYASVYEEKSYTDGRRENPCEFNRGYRDYTPLPVGYVTKELEYFPETDKVW | |
| | IEIGETEGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 9 |
| 020 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYYTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKAVNA | |
| | LFTSTNQLGLKTNVTDYHIDQVSNLVTYLSDEFCLDEKRELSEKVKHAKRLSDE | |
| | RNLLQDPNFRGINRQPDRGWRGSTDITIQGGDDVFKENYVTLSGTFDECYPTYL | |
| | YQKIDESKLKAYTRYQLRGYIEDSQDLEIYLIRYNAKHETVNVPGTGSLWPLSV | |
| | ENQIGPCGEPNRCAPHLEWNPDLHCSCRDGEKCAHHSHHFSLDIDVGCTDLNE | |
| | DLGVWVIFKIKTQDGHARLGNLEFLEEKPLVGEALARVKRAEKKWRDKREKL | |
| | EWETNIVYKEAKESVDALFVNSQYDQLQADTNIAMIHAADKRVHSIREAYLPE | |
| | LSVIPGVNAAIFEELEGRIFTAFSLYDARNVIKNGDENNGLSCWNVKGHVDVEE | |
| | QNNQRSVLVVPEWEAEVSQEVRVCPGRGYILRVTAYKEGYGEGCVTIHEIENN | |
| | TDELKFSNCVEEEIYPNNTVTCNDYTVNQEEYGGAYTSRNRGYNEAYGNNPSV | |
| | PADYASVYEEKSYTDGRRENPCEFNRGYRDYTPLPVGYVTKELEYFPETDKVW | |
| | IEIGETEGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 10 |
| 021 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYWTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKAVNA | |
| | LFTSTNQLGLKTNVTDYHIDQVSNLVTYLSDEFCLDEKRELSEKVKHAKRLSDE | |
| | RNLLQDPNFRGINRQPDRGWRGSTDITIQGGDDVFKENYVTLSGTFDECYPTYL | |
| | YQKIDESKLKAYTRYQLRGYIEDSQDLEIYLIRYNAKHETVNVPGTGSLWPLSV | |
| | ENQIGPCGEPNRCAPHLEWNPDLHCSCRDGEKCAHHSHHFSLDIDVGCTDLNE | |
| | DLGVWVIFKIKTQDGHARLGNLEFLEEKPLVGEALARVKRAEKKWRDKREKL | |
| | EWETNIVYKEAKESVDALFVNSQYDQLQADTNIAMIHAADKRVHSIREAYLPE | |
| | LSVIPGVNAAIFEELEGRIFTAFSLYDARNVIKNGDFNNGLSCWNVKGHVDVEE | |
| | QNNQRSVLVVPEWEAEVSQEVRVCPGRGYILRVTAYKEGYGEGCVTIHEIENN | |
| | TDELKFSNCVEEEIYPNNTVTCNDYTVNQEEYGGAYTSRNRGYNEAYGNNPSV | |
| | PADYASVYEEKSYTDGRRENPCEFNRGYRDYTPLPVGYVTKELEYFPETDKVW | |
| | IEIGETEGTFIVDSVELLLMEE | |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 11 |
| 022 | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYFTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKAVNA | |
| | LFTSTNQLGLKTNVTDYHIDQVSNLVTYLSDEFCLDEKRELSEKVKHAKRLSDE | |
| | RNLLQDSNFKDINRQPERGWGGSTGITIQGGDDVFKENYVTLSGTFDECYPTYL | |
| | YQKIDESKLKAFTRYQLRGYIEDSQDLEIYSIRYNAKHETVNVPGTGSLWPLSA | |
| | QSPIGKCGEPNRCAPHLEWNPDLDCSCRDGEKCAHHSHHFSLDIDVGCTDLNE | |
| | DLGVWVIFKIKTQDGHARLGNLEFLEEKPLVGEALARVKRAEKKWRDKREKL | |
| | EWETNIVYKEAKESVDALFVNSQYDQLQADTNIAMIHAADKRVHSIREAYLPE | |
| | LSVIPGVNAAIFEELEGRIFTAFSLYDARNVIKNGDFNNGLSCWNVKGHVDVEE | |
| | QNNQRSVLVVPEWEAEVSQEVRVCPGRGYILRVTAYKEGYGEGCVTIHEIENN | |
| | TDELKFSNCVEEEIYPNNTVTCNDYTVNQEEYGGAYTSRNRGYNEAPSVPADY | |
| | ASVYEEKSYTDGRRENPCEFNRGYRDYTPLPVGYVTKELEYFPETDKVWIEIGE | |
| | TEGTFIVDSVELLLMEE | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 12 |
| encoding | tttagcgactgggagaaagatccgaccaatccagcactgcgtgaagagatgcggattcagttcaatgacatgaacagcgcattg | |
| BD1-001 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcatcctatccaaccttttctgccgctgaaagcgcaataattcgtagtccacatctggttgactttttaaattccttcacaatc | |
| | tataccgatagcctagcacggtatgcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctatattacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagttgtgaatgcgttgtttactagcaccaac | |
| | caacttggactcaaaacagatgttaccgactaccatatagatcaggtttccaacctcgttgcgtgtctcagcgatgagttctgt | |
| | ttggatgaaaaaagagaactgtcagagaaagttaaacatgcgaaacgtctcagcgacgaacgaaatctgctgcaagatcccaat | |
| | tttcgtggtattaatcgtcagcctgatcgtgggtggcgtggttccacggatatcaccattcagggtggtgatgatgttttcaag | |
| | gaaaattatgttacactgccaggaacctttgatgagtgctacccgacatatctgtaccagaagattgatgaatcaaaactgaaa | |
| | gcatatacaagatatcagctccgtgggtacatagaggactctcaggatctagagatttatctaatccgctataatgcgaagcat | |
| | gaaatagttaatgttcctggtactggatctctgtggccgctgagcgttgagaatcagattggtccttgtggagagcctaataga | |
| | tgtgcaccgcacctcgagtggaaccccgacctccattgctcgtgtcgtgatggagagaaatgtgcacatcacagccaccatttc | |
| | agcctggatattgacgttggctgcacagatctgaatgaagacctcggcgtgtgggttatctttaaaattaaaactcaggatggt | |
| | catgcaagactcggcaatctcgaatttctggaggaaaaaccgctgctgggtgaagcattagctcgtgtgaagcgtgcagaaaag | |
| | aagtggagagataaacgtgagacactgcagctggaaactaccattgtgtacaaagaagcaaaagaaagcgttgatgcgctcttt | |
| | gtaaactcgcagtacgatcgtctgcaggcagatacaaacattgccatgattcatgctgcggataaacgtgtacaccgaattcgt | |
| | gaagcatatctcccagaactgagcgtgatcccgggcgtgaatgcagcaatctttgaagaattggaagaacgtatatttactgcc | |
| | ttctctttgtatgacgcacggaacattattaaaaatggagatttcaacaatggcctcctgtgctggaatgtgaaaggccatgtg | |
| | gaagttgaggagcagaataaccatcggtccgttctggttattcctgaatgggaagccgaggtttcacaagaagttcgcgtctgt | |
| | ccgggtcgtggttacattctgcgcgttaccgcgtacaaagaaggttatggtgaaggttgcgttacaatccatgagatcgagaac | |
| | aatacagatgaactgaaattcaataattgcgtcgaagaggaggtatacccaaacaatactgttacgtgcataaattacaccgca | |
| | acgcaggaggagtacgaaggtacgtataccagccgcaatcgtggctatgacgaagcatatggaaacaacccgagcgttccggcg | |
| | gattatgctagcgtctatgaagaaaaatcttacaccgatcgtcgtcgcgagaacccgtgtgaaagcaacaggggttacggagat | |
| | tataccccgctgcccgcgggctatgtaaccaaagagctggaatattttccggagaccgacaaagtgtggattgaaattggcgaa | |
| | actgaaggcacctttatcgtagattctgtagaactgctgctgatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 13 |
| encoding | tttagcgactgggagaaagatccgaccaatccagcactgcgtgaagagatgcggattcagttcaatgacatgaacagcgcattg | |
| BD1-002 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcagtttatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctatccgacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagttgtgaatgcgttgtttactagcaccaac | |
| | caacttggactcaaaacagatgttaccgactaccatatagatcaggtttccaacctcgttgcgtgtctcagcgatgagttctgt | |
| | ttggatgaaaaaagagaactgtcagagaaagttaaacatgcgaaacgtctcagcgacgaacgaaatctgctgcaagatcccaat | |
| | tttcgtggtattaatcgtcagcctgatcgtgggtggcgtggttccacggatatcaccattcagggtggtgatgatgttttcaag | |
| | gaaaattatgttacactgccaggaacctttgatgagtgctacccgacatatctgtaccagaagattgatgaatcaaaactgaaa | |
| | gcatatacaagatatcagctccgtgggtacatagaggactctcaggatctagagatttatctaatccgctataatgcgaagcat | |
| | gaaatagttaatgttcctggtactggatctctgtggccgctgagcgttgagaatcagattggtccttgtggagagcctaataga | |
| | tgtgcaccgcacctcgagtggaaccccgacctccattgctcgtgtcgtgatggagagaaatgtgcacatcacagccaccatttc | |
| | agcctggatattgacgttggctgcacagatctgaatgaagacctcggcgtgtgggttatctttaaaattaaaactcaggatggt | |
| | catgcaagactcggcaatctcgaatttctggaggaaaaaccgctgctgggtgaagcattagctcgtgtgaagcgtgcagaaaag | |
| | aagtggagagataaacgtgagacactgcagctggaaactaccattgtgtacaaagaagcaaaagaaagcgttgatgcgctcttt | |
| | gtaaactcgcagtacgatcgtctgcaggcagatacaaacattgccatgattcatgctgcggataaacgtgtacaccgaattcgt | |
| | gaagcatatctcccagaactgagcgtgatcccgggcgtgaatgcagcaatctttgaagaattggaagaacgtatatttactgcc | |
| | ttctctttgtatgacgcacggaacattattaaaaatggagatttcaacaatggcctcctgtgctggaatgtgaaaggccatgtg | |
| | gaagttgaggagcagaataaccatcggtccgttctggttattcctgaatgggaagccgaggtttcacaagaagttcgcgtctgt | |
| | ccgggtcgtggttacattctgcgcgttaccgcgtacaaagaaggttatggtgaaggttgcgttacaatccatgagatcgagaac | |
| | aatacagatgaactgaaattcaataattgcgtcgaagaggaggtatacccaaacaatactgttacgtgcataaattacaccgca | |
| | acgcaggaggagtacgaaggtacgtataccagccgcaatcgtggctatgacgaagcatatggaaacaacccgagcgttccggcg | |
| | gattatgctagcgtctatgaagaaaaatcttacaccgatcgtcgtcgcgagaacccgtgtgaaagcaacaggggttacggagat | |
| | tataccccgctgcccgcgggctatgtaaccaaagagctggaatattttccggagaccgacaaagtgtggattgaaattggcgaa | |
| | actgaaggcacctttatcgtagattctgtagaactgctgctgatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 14 |
| encoding | tttagcgactgggagaaagatccgaccaatccagcactgcgtgaagagatgcggattcagttcaatgacatgaacagcgcattg | |
| BD1-011 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcactgtatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctatccgacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagttgtgaatgcgttgtttactagcaccaac | |
| | caacttggactcaaaacagatgttaccgactaccatatagatcaggtttccaacctcgttgcgtgtctcagcgatgagttctgt | |
| | ttggatgaaaaaagagaactgtcagagaaagttaaacatgcgaaacgtctcagcgacgaacgaaatctgctgcaagatcccaat | |
| | tttcgtggtattaatcgtcagcctgatcgtgggtggcgtggttccacggatatcaccattcagggtggtgatgatgttttcaag | |
| | gaaaattatgttacactgccaggaacctttgatgagtgctacccgacatatctgtaccagaagattgatgaatcaaaactgaaa | |
| | gcatatacaagatatcagctccgtgggtacatagaggactctcaggatctagagatttatctaatccgctataatgcgaagcat | |
| | gaaatagttaatgttcctggtactggatctctgtggccgctgagcgttgagaatcagattggtccttgtggagagcctaataga | |
| | tgtgcaccgcacctcgagtggaaccccgacctccattgctcgtgtcgtgatggagagaaatgtgcacatcacagccaccatttc | |
| | agcctggatattgacgttggctgcacagatctgaatgaagacctcggcgtgtgggttatctttaaaattaaaactcaggatggt | |
| | catgcaagactcggcaatctcgaatttctggaggaaaaaccgctgctgggtgaagcattagctcgtgtgaagcgtgcagaaaag | |
| | aagtggagagataaacgtgagacactgcagctggaaactaccattgtgtacaaagaagcaaaagaaagcgttgatgcgctcttt | |
| | gtaaactcgcagtacgatcgtctgcaggcagatacaaacattgccatgattcatgctgcggataaacgtgtacaccgaattcgt | |
| | gaagcatatctcccagaactgagcgtgatcccgggcgtgaatgcagcaatctttgaagaattggaagaacgtatatttactgcc | |
| | ttctctttgtatgacgcacggaacattattaaaaatggagatttcaacaatggcctcctgtgctggaatgtgaaaggccatgtg | |
| | gaagttgaggagcagaataaccatcggtccgttctggttattcctgaatgggaagccgaggtttcacaagaagttcgcgtctgt | |
| | ccgggtcgtggttacattctgcgcgttaccgcgtacaaagaaggttatggtgaaggttgcgttacaatccatgagatcgagaac | |
| | aatacagatgaactgaaattcaataattgcgtcgaagaggaggtatacccaaacaatactgttacgtgcataaattacaccgca | |
| | acgcaggaggagtacgaaggtacgtataccagccgcaatcgtggctatgacgaagcatatggaaacaacccgagcgttccggcg | |
| | gattatgctagcgtctatgaagaaaaatcttacaccgatcgtcgtcgcgagaacccgtgtgaaagcaacaggggttacggagat | |
| | tataccccgctgcccgcgggctatgtaaccaaagagctggaatattttccggagaccgacaaagtgtggattgaaattggcgaa | |
| | actgaaggcacctttatcgtagattctgtagaactgctgctgatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 15 |
| encoding | tttagcgactgggagaaagatccgaccaatccagcactgcgtgaagagatgcggattcagttcaatgacatgaacagcgcattg | |
| BD1-013 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcagtttatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctattttacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagttgtgaatgcgttgtttactagcaccaac | |
| | caacttggactcaaaacagatgttaccgactaccatatagatcaggtttccaacctcgttgcgtgtctcagcgatgagttctgt | |
| | ttggatgaaaaaagagaactgtcagagaaagttaaacatgcgaaacgtctcagcgacgaacgaaatctgctgcaagatcccaat | |
| | tttcgtggtattaatcgtcagcctgatcgtgggtggcgtggttccacggatatcaccattcagggtggtgatgatgttttcaag | |
| | gaaaattatgttacactgccaggaacctttgatgagtgctacccgacatatctgtaccagaagattgatgaatcaaaactgaaa | |
| | gcatatacaagatatcagctccgtgggtacatagaggactctcaggatctagagatttatctaatccgctataatgcgaagcat | |
| | gaaatagttaatgttcctggtactggatctctgtggccgctgagcgttgagaatcagattggtccttgtggagagcctaataga | |
| | tgtgcaccgcacctcgagtggaaccccgacctccattgctcgtgtcgtgatggagagaaatgtgcacatcacagccaccatttc | |
| | agcctggatattgacgttggctgcacagatctgaatgaagacctcggcgtgtgggttatctttaaaattaaaactcaggatggt | |
| | catgcaagactcggcaatctcgaatttctggaggaaaaaccgctgctgggtgaagcattagctcgtgtgaagcgtgcagaaaag | |
| | aagtggagagataaacgtgagacactgcagctggaaactaccattgtgtacaaagaagcaaaagaaagcgttgatgcgctcttt | |
| | gtaaactcgcagtacgatcgtctgcaggcagatacaaacattgccatgattcatgctgcggataaacgtgtacaccgaattcgt | |
| | gaagcatatctcccagaactgagcgtgatcccgggcgtgaatgcagcaatctttgaagaattggaagaacgtatatttactgcc | |
| | ttctctttgtatgacgcacggaacattattaaaaatggagatttcaacaatggcctcctgtgctggaatgtgaaaggccatgtg | |
| | gaagttgaggagcagaataaccatcggtccgttctggttattcctgaatgggaagccgaggtttcacaagaagttcgcgtctgt | |
| | ccgggtcgtggttacattctgcgcgttaccgcgtacaaagaaggttatggtgaaggttgcgttacaatccatgagatcgagaac | |
| | aatacagatgaactgaaattcaataattgcgtcgaagaggaggtatacccaaacaatactgttacgtgcataaattacaccgca | |
| | acgcaggaggagtacgaaggtacgtataccagccgcaatcgtggctatgacgaagcatatggaaacaacccgagcgttccggcg | |
| | gattatgctagcgtctatgaagaaaaatcttacaccgatcgtcgtcgcgagaacccgtgtgaaagcaacaggggttacggagat | |
| | tataccccgctgcccgcgggctatgtaaccaaagagctggaatattttccggagaccgacaaagtgtggattgaaattggcgaa | |
| | actgaaggcacctttatcgtagattctgtagaactgctgctgatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 16 |
| encoding | tttagcgactgggagaaagatccgaccaatccagcactgcgtgaagagatgcggattcagttcaatgacatgaacagcgcattg | |
| BD1-014 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcactgtatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctattttacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagttgtgaatgcgttgtttactagcaccaac | |
| | caacttggactcaaaacagatgttaccgactaccatatagatcaggtttccaacctcgttgcgtgtctcagcgatgagttctgt | |
| | ttggatgaaaaaagagaactgtcagagaaagttaaacatgcgaaacgtctcagcgacgaacgaaatctgctgcaagatcccaat | |
| | tttcgtggtattaatcgtcagcctgatcgtgggtggcgtggttccacggatatcaccattcagggtggtgatgatgttttcaag | |
| | gaaaattatgttacactgccaggaacctttgatgagtgctacccgacatatctgtaccagaagattgatgaatcaaaactgaaa | |
| | gcatatacaagatatcagctccgtgggtacatagaggactctcaggatctagagatttatctaatccgctataatgcgaagcat | |
| | gaaatagttaatgttcctggtactggatctctgtggccgctgagcgttgagaatcagattggtccttgtggagagcctaataga | |
| | tgtgcaccgcacctcgagtggaaccccgacctccattgctcgtgtcgtgatggagagaaatgtgcacatcacagccaccatttc | |
| | agcctggatattgacgttggctgcacagatctgaatgaagacctcggcgtgtgggttatctttaaaattaaaactcaggatggt | |
| | catgcaagactcggcaatctcgaatttctggaggaaaaaccgctgctgggtgaagcattagctcgtgtgaagcgtgcagaaaag | |
| | aagtggagagataaacgtgagacactgcagctggaaactaccattgtgtacaaagaagcaaaagaaagcgttgatgcgctcttt | |
| | gtaaactcgcagtacgatcgtctgcaggcagatacaaacattgccatgattcatgctgcggataaacgtgtacaccgaattcgt | |
| | gaagcatatctcccagaactgagcgtgatcccgggcgtgaatgcagcaatctttgaagaattggaagaacgtatatttactgcc | |
| | ttctctttgtatgacgcacggaacattattaaaaatggagatttcaacaatggcctcctgtgctggaatgtgaaaggccatgtg | |
| | gaagttgaggagcagaataaccatcggtccgttctggttattcctgaatgggaagccgaggtttcacaagaagttcgcgtctgt | |
| | ccgggtcgtggttacattctgcgcgttaccgcgtacaaagaaggttatggtgaaggttgcgttacaatccatgagatcgagaac | |
| | aatacagatgaactgaaattcaataattgcgtcgaagaggaggtatacccaaacaatactgttacgtgcataaattacaccgca | |
| | acgcaggaggagtacgaaggtacgtataccagccgcaatcgtggctatgacgaagcatatggaaacaacccgagcgttccggcg | |
| | gattatgctagcgtctatgaagaaaaatcttacaccgatcgtcgtcgcgagaacccgtgtgaaagcaacaggggttacggagat | |
| | tataccccgctgcccgcgggctatgtaaccaaagagctggaatattttccggagaccgacaaagtgtggattgaaattggcgaa | |
| | actgaaggcacctttatcgtagattctgtagaactgctgctgatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 17 |
| encoding | tttagcgactgggagaaagatccgaccaatccagcactgcgtgaagagatgcggattcagttcaatgacatgaacagcgcattg | |
| BD1-015 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcagtttatccaaccttttctgccgctgaaagcgcaataattcgtagtccacatctggttgactttttaaattccttcacaatc | |
| | tataccgatagcctagcacggaccgcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctatccgacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagttgtgaatgcgttgtttactagcaccaac | |
| | caacttggactcaaaacagatgttaccgactaccatatagatcaggtttccaacctcgttgcgtgtctcagcgatgagttctgt | |
| | ttggatgaaaaaagagaactgtcagagaaagttaaacatgcgaaacgtctcagcgacgaacgaaatctgctgcaagatcccaat | |
| | tttcgtggtattaatcgtcagcctgatcgtgggtggcgtggttccacggatatcaccattcagggtggtgatgatgttttcaag | |
| | gaaaattatgttacactgccaggaacctttgatgagtgctacccgacatatctgtaccagaagattgatgaatcaaaactgaaa | |
| | gcatatacaagatatcagctccgtgggtacatagaggactctcaggatctagagatttatctaatccgctataatgcgaagcat | |
| | gaaatagttaatgttcctggtactggatctctgtggccgctgagcgttgagaatcagattggtccttgtggagagcctaataga | |
| | tgtgcaccgcacctcgagtggaaccccgacctccattgctcgtgtcgtgatggagagaaatgtgcacatcacagccaccatttc | |
| | agcctggatattgacgttggctgcacagatctgaatgaagacctcggcgtgtgggttatctttaaaattaaaactcaggatggt | |
| | catgcaagactcggcaatctcgaatttctggaggaaaaaccgctgctgggtgaagcattagctcgtgtgaagcgtgcagaaaag | |
| | aagtggagagataaacgtgagacactgcagctggaaactaccattgtgtacaaagaagcaaaagaaagcgttgatgcgctcttt | |
| | gtaaactcgcagtacgatcgtctgcaggcagatacaaacattgccatgattcatgctgcggataaacgtgtacaccgaattcgt | |
| | gaagcatatctcccagaactgagcgtgatcccgggcgtgaatgcagcaatctttgaagaattggaagaacgtatatttactgcc | |
| | ttctctttgtatgacgcacggaacattattaaaaatggagatttcaacaatggcctcctgtgctggaatgtgaaaggccatgtg | |
| | gaagttgaggagcagaataaccatcggtccgttctggttattcctgaatgggaagccgaggtttcacaagaagttcgcgtctgt | |
| | ccgggtcgtggttacattctgcgcgttaccgcgtacaaagaaggttatggtgaaggttgcgttacaatccatgagatcgagaac | |
| | aatacagatgaactgaaattcaataattgcgtcgaagaggaggtatacccaaacaatactgttacgtgcataaattacaccgca | |
| | acgcaggaggagtacgaaggtacgtataccagccgcaatcgtggctatgacgaagcatatggaaacaacccgagcgttccggcg | |
| | gattatgctagcgtctatgaagaaaaatcttacaccgatcgtcgtcgcgagaacccgtgtgaaagcaacaggggttacggagat | |
| | tataccccgctgcccggggctatgtaaccaaagagctggaatattttccggagaccgacaaagtgtggattgaaattggcgaaa | |
| | ctgaaggcacctttatcgtagattctgtagaactgctgctgatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 18 |
| encoding | tttagcgactgggagaaagatccgaccaatccagcactgcgtgaagagatgcggattcagttcaatgacatgaacagcgcattg | |
| BD1-018 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcagtttatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctattttacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagccgtgaacgcattgtttacaagcaccaac | |
| | caactgggtctgaaaacgaatgtaaccgactaccacatagatcaggtcagtaatctggtaacatatctgtccgatgagttctgc | |
| | ctcgatgaaaagagggaactgtctgaaaaggtcaaacatgccaaaagactgagcgatgagaggaacctccttcaggacccgaac | |
| | tttcggggaataaatcgccagccggaccgtggatggcgtggtagcaccgacattaccatccagggcggcgacgatgtgttcaag | |
| | gaaaactacgtcaccctgagcggtacgtttgatgagtgctacccgacttacctttatcagaagatagatgaaagcaagctcaag | |
| | gcatacactcgctatcagctccgcgggtacattgaggattcacaagatcttgagatttatctcatcagatacaacgcaaaacat | |
| | gaaaccgttaatgtgccgggcaccggtagcctctggccgctgagcgttgaaaatcaaattggcccgtgtggcgagccgaaccgg | |
| | tgtgcgccacatttggagtggaatccggatctgcactgctcttgtagggacggggaaaaatgtgcccatcattcacatcacttt | |
| | agcctggatattgatgttgggtgtaccgatttaaatgaagatctaggtgtctgggtgatcttcaaaattaaaactcaggacggc | |
| | cacgcacggctgggtaacttggaatttctagaagagaaacccctggtcggagaggcgctggcccgtgttaaacgggcagaaaag | |
| | aagtggcgtgacaaacgtgagaaactggaatgggagacgaacattgtgtacaaggaggcaaaagaaagcgttgatgcgctcttt | |
| | gtgaattctcagtacgaccagctgcaggcagatactaatattgccatgatacatgctgcagacaagcgtgttcattccattcgc | |
| | gaggcctacctgccggagctgagcgttattcctggagtcaatgctgcaatatttgaggagttggaaggtcgtattttcaccgcc | |
| | ttctctttgtatgatgctcgtaatgtcatcaagaacggtgacttcaacaatggcctgagctgctggaacgttaaagggcatgtt | |
| | gatgttgaagagcagaacaatcaaagatccgttctggtggtcccggagtgggaagccgaagttagccaggaggtgcgagtttgc | |
| | ccgggtcgtggctatattctacgtgtcaccgcatataaagagggttatggtgaaggatgtgttactatccatgagatcgaaaat | |
| | aataccgatgaactgaagttttcgaattgcgtggaggaagaaatctatcctaataatacagttacctgcaacgattatacggtt | |
| | aaccaggaagaatatggtggtgcatataccagccgcaatcgtggttataacgaagcatatgggaataacccgtcggttccagca | |
| | gattatgctagcgtatatgaggaaaaatcttataccgatggtagacgtgaaaacccatgtgagttcaatcgtggttatcgtgat | |
| | tacacgccgctaccagtaggttatgttactaaagagctggagtactttccagaaacggacaaggtttggatcgaaattggtgaa | |
| | acagaaggtacatttattgtcgacagcgtcgaactgctgctcatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 19 |
| encoding | tttagcgactgggagaaagatccgaccaatccagcactgcgtgaagagatgcggattcagttcaatgacatgaacagcgcattg | |
| BD1-019 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcagtttatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctattttacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctaccctggaggcggagtataacctagaaagagctcaaaaagccgtgaacgcattgtttacaagcaccaac | |
| | caactgggtctgaaaacgaatgtaaccgactaccacatagatcaggtcagtaatctggtaacatatctgtccgatgagttctgc | |
| | ctcgatgaaaagagggaactgtctgaaaaggtcaaacatgccaaaagactgagcgatgagaggaacctccttcaggacccgaac | |
| | tttcggggaataaatcgccagccggaccgtggatggcgtggtagcaccgacattaccatccagggcggcgacgatgtgttcaag | |
| | gaaaactacgtcaccctgagcggtacgtttgatgagtgctacccgacttacctttatcagaagatagatgaaagcaagctcaag | |
| | gcatacactcgctatcagctccgcgggtacattgaggattcacaagatcttgagatttatctcatcagatacaacgcaaaacat | |
| | gaaaccgttaatgtgccgggcaccggtagcctctggccgctgagcgttgaaaatcaaattggcccgtgtggcgagccgaaccgg | |
| | tgtgcgccacatttggagtggaatccggatctgcactgctcttgtagggacggggaaaaatgtgcccatcattcacatcacttt | |
| | agcctggatattgatgttgggtgtaccgatttaaatgaagatctaggtgtctgggtgatcttcaaaattaaaactcaggacggc | |
| | cacgcacggctgggtaacttggaatttctagaagagaaacccctggtcggagaggcgctggcccgtgttaaacgggcagaaaag | |
| | aagtggcgtgacaaacgtgagaaactggaatgggagacgaacattgtgtacaaggaggcaaaagaaagcgttgatgcgctcttt | |
| | gtgaattctcagtacgaccagctgcaggcagatactaatattgccatgatacatgctgcagacaagcgtgttcattccattcgc | |
| | gaggcctacctgccggagctgagcgttattcctggagtcaatgctgcaatatttgaggagttggaaggtcgtattttcaccgcc | |
| | ttctctttgtatgatgctcgtaatgtcatcaagaacggtgacttcaacaatggcctgagctgctggaacgttaaagggcatgtt | |
| | gatgttgaagagcagaacaatcaaagatccgttctggtggtcccggagtgggaagccgaagttagccaggaggtgcgagtttgc | |
| | ccgggtcgtggctatattctacgtgtcaccgcatataaagagggttatggtgaaggatgtgttactatccatgagatcgaaaat | |
| | aataccgatgaactgaagttttcgaattgcgtggaggaagaaatctatcctaataatacagttacctgcaacgattatacggtt | |
| | aaccaggaagaatatggtggtgcatataccagccgcaatcgtggttataacgaagcatatgggaataacccgtcggttccagca | |
| | gattatgctagcgtatatgaggaaaaatcttataccgatggtagacgtgaaaacccatgtgagttcaatcgtggttatcgtgat | |
| | tacacgccgctaccagtaggttatgttactaaagagctggagtactttccagaaacggacaaggtttggatcgaaattggtgaa | |
| | acagaaggtacatttattgtcgacagcgtcgaactgctgctcatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 20 |
| encoding | tttagcgactgggagaaagatccgaccaatccagcactgcgtgaagagatgcggattcagttcaatgacatgaacagcgcattg | |
| BD1-020 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcagtttatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctattatacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagccgtgaacgcattgtttacaagcaccaac | |
| | caactgggtctgaaaacgaatgtaaccgactaccacatagatcaggtcagtaatctggtaacatatctgtccgatgagttctgc | |
| | ctcgatgaaaagagggaactgtctgaaaaggtcaaacatgccaaaagactgagcgatgagaggaacctccttcaggacccgaac | |
| | tttcggggaataaatcgccagccggaccgtggatggcgtggtagcaccgacattaccatccagggcggcgacgatgtgttcaag | |
| | gaaaactacgtcaccctgagcggtacgtttgatgagtgctacccgacttacctttatcagaagatagatgaaagcaagctcaag | |
| | gcatacactcgctatcagctccgcgggtacattgaggattcacaagatcttgagatttatctcatcagatacaacgcaaaacat | |
| | gaaaccgttaatgtgccgggcaccggtagcctctggccgctgagcgttgaaaatcaaattggcccgtgtggcgagccgaaccgg | |
| | tgtgcgccacatttggagtggaatccggatctgcactgctcttgtagggacggggaaaaatgtgcccatcattcacatcacttt | |
| | agcctggatattgatgttgggtgtaccgatttaaatgaagatctaggtgtctgggtgatcttcaaaattaaaactcaggacggc | |
| | cacgcacggctgggtaacttggaatttctagaagagaaacccctggtcggagaggcgctggcccgtgttaaacgggcagaaaag | |
| | aagtggcgtgacaaacgtgagaaactggaatgggagacgaacattgtgtacaaggaggcaaaagaaagcgttgatgcgctcttt | |
| | gtgaattctcagtacgaccagctgcaggcagatactaatattgccatgatacatgctgcagacaagcgtgttcattccattcgc | |
| | gaggcctacctgccggagctgagcgttattcctggagtcaatgctgcaatatttgaggagttggaaggtcgtattttcaccgcc | |
| | ttctctttgtatgatgctcgtaatgtcatcaagaacggtgacttcaacaatggcctgagctgctggaacgttaaagggcatgtt | |
| | gatgttgaagagcagaacaatcaaagatccgttctggtggtcccggagtgggaagccgaagttagccaggaggtgcgagtttgc | |
| | ccgggtcgtggctatattctacgtgtcaccgcatataaagagggttatggtgaaggatgtgttactatccatgagatcgaaaat | |
| | aataccgatgaactgaagttttcgaattgcgtggaggaagaaatctatcctaataatacagttacctgcaacgattatacggtt | |
| | aaccaggaagaatatggtggtgcatataccagccgcaatcgtggttataacgaagcatatgggaataacccgtcggttccagca | |
| | gattatgctagcgtatatgaggaaaaatcttataccgatggtagacgtgaaaacccatgtgagttcaatcgtggttatcgtgat | |
| | tacacgccgctaccagtaggttatgttactaaagagctggagtactttccagaaacggacaaggtttggatcgaaattggtgaa | |
| | acagaaggtacatttattgtcgacagcgtcgaactgctgctcatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggggcggttttatag | ID |
| tide | taggcctgctagagcttatctggggatttatcggtccgtcccagtgggatatctttctggcccagattgagcagctgatttcac | NO: |
| sequence | agaggattgaagaatttgctagaaaccaagcaatttccaggctcgaagggttatctaatttgtataaagtatatgtacgggctt | 21 |
| encoding | ttagcgactgggagaaagatccgaccaatccagcactgcgtgaagagatgcggattcagttcaatgacatgaacagcgcattga | |
| BD1-021 | tcaccgcaattccgctcttccgcgtacaaaattatgaagtggcgttgctgtccgtctatgttcaagccgctaacttacacctaa | |
| | gcattctgcgggatgtgagtgtttttggcgaacgatggggttatgataccgcgacgatcaataatagatattccgatctgacca | |
| | gcctcatccatgtttatacaaaccattgtgttgatacgtataaccagggactcagacgtctggagggtcggttcttatccgatt | |
| | ggatcgtatataaccgttttagacgccagttgacaattagcgttctggatattgtggcgttcttcccaaattatgatattcgta | |
| | cctatccgatacagactgcgactcagctgacgagagaagtttatcttgatcttccgttcatcaacgagaatctgtctcctgcag | |
| | cagtttatccaaccttttctgccgctgaaagcgcaataattcgtagtccacatctggttgactttttaaattccttcacaatct | |
| | ataccgatagcctagcacggtcagcatactggggaggccacctggtgaatagttttcgtacgggcaccaccaccaatttgattc | |
| | gttccccgttgtatggcagggaaggcaacacagaaaggccagttaccatcaccgcaagcccgagcgttcccatttttcgtactc | |
| | tgagctattggacgggtctggacaacagcaaccctgttgctgggattgagggtgttgagttccagaataccattagccgtagta | |
| | tctacagaaaaagcggacctatcgattcctttagcgaactgcccccgcaggatgcatctgtgagcccggccattggctatagcc | |
| | accgtctgtgccacgccacgtttctggagcgtatcagcgggccgcgaatcgctggtactgtgttttcctggacccatcgtagcg | |
| | ccagtccgaccaatgaagttagcccttctcgtatcacgcaaatcccatgggtgaaggcacataccctcgcatcgggtgcatccg | |
| | tcattaagggcccgggctttaccggtggggacattctgaccagaaactccatgggtgaactcggcaccctgcgcgtaacattta | |
| | ctggtaggctgccccagagctactacattcgatttcgttatgccagcgtcgccaaccggagcggcacctttcgatattcccagc | |
| | caccgtcctatggcatatcatttccaaaaaccatggacgcaggtgagcctctcactagccgttcgtttgcacataccaccctgt | |
| | tcacgccgattacgttttcccgtgcacaggaagaattcgacctctatatccagagtggcgtttacatcgaccgcatcgaattta | |
| | ttccggttacggctacctttgaggcggagtatgatctagaaagagctcaaaaagccgtgaacgcattgtttacaagcaccaacc | |
| | aactgggtctgaaaacgaatgtaaccgactaccacatagatcaggtcagtaatctggtaacatatctgtccgatgagttctgcc | |
| | tcgatgaaaagagggaactgtctgaaaaggtcaaacatgccaaaagactgagcgatgagaggaacctccttcaggacccgaact | |
| | ttcggggaataaatcgccagccggaccgtggatggcgtggtagcaccgacattaccatccagggcggcgacgatgtgttcaagg | |
| | aaaactacgtcaccctgagcggtacgtttgatgagtgctacccgacttacctttatcagaagatagatgaaagcaagctcaagg | |
| | catacactcgctatcagctccgcgggtacattgaggattcacaagatcttgagatttatctcatcagatacaacgcaaaacatg | |
| | aaaccgttaatgtgccgggcaccggtagcctctggccgctgagcgttgaaaatcaaattggcccgtgtggcgagccgaaccggt | |
| | gtgcgccacatttggagtggaatccggatctgcactgctcttgtagggacggggaaaaatgtgcccatcattcacatcacttta | |
| | gcctggatattgatgttgggtgtaccgatttaaatgaagatctaggtgtctgggtgatcttcaaaattaaaactcaggacggcc | |
| | acgcacggctgggtaacttggaatttctagaagagaaacccctggtcggagaggcgctggcccgtgttaaacgggcagaaaaga | |
| | agtggcgtgacaaacgtgagaaactggaatgggagacgaacattgtgtacaaggaggcaaaagaaagcgttgatgcgctctttg | |
| | tgaattctcagtacgaccagctgcaggcagatactaatattgccatgatacatgctgcagacaagcgtgttcattccattcgcg | |
| | aggcctacctgccggagctgagcgttattcctggagtcaatgctgcaatatttgaggagttggaaggtcgtattttcaccgcct | |
| | tctctttgtatgatgctcgtaatgtcatcaagaacggtgacttcaacaatggcctgagctgctggaacgttaaagggcatgttg | |
| | atgttgaagagcagaacaatcaaagatccgttctggtggtcccggagtgggaagccgaagttagccaggaggtgcgagtttgcc | |
| | cgggtcgtggctatattctacgtgtcaccgcatataaagagggttatggtgaaggatgtgttactatccatgagatcgaaaata | |
| | ataccgatgaactgaagttttcgaattgcgtggaggaagaaatctatcctaataatacagttacctgcaacgattatacggtta | |
| | accaggaagaatatggtggtgcatataccagccgcaatcgtggttataacgaagcatatgggaataacccgtcggttccagcag | |
| | attatgctagcgtatatgaggaaaaatcttataccgatggtagacgtgaaaacccatgtgagttcaatcgtggttatcgtgatt | |
| | acacgccgctaccagtaggttatgttactaaagagctggagtactttccagaaacggacaaggtttggatcgaaattggtgaaa | |
| | cagaaggtacatttattgtcgacagcgtcgaactgctgctcatggaggaataa | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggtggcggttttata | ID |
| tide | gtaggcctgctagagcttatctggggatttatcggtccgtcccagtgggatatctttctggcccagattgagcagctgatttca | NO: |
| sequence | cagaggattgaagaatttgctagaaaccaagcaatttccaggctcgaagggttatctaatttgtataaagtatatgtacgggct | 22 |
| encoding | tttagcgactgggagaaagatccgaccaatccagcactgcgtgaagagatgcggattcagttcaatgacatgaacagcgcattg | |
| BD1-022 | atcaccgcaattccgctcttccgcgtacaaaattatgaagtggcgttgctgtccgtctatgttcaagccgctaacttacaccta | |
| | agcattctgcgggatgtgagtgtttttggcgaacgatggggttatgataccgcgacgatcaataatagatattccgatctgacc | |
| | agcctcatccatgtttatacaaaccattgtgttgatacgtataaccagggactcagacgtctggagggtcggttcttatccgat | |
| | tggatcgtatataaccgttttagacgccagttgacaattagcgttctggatattgtggcgttcttcccaaattatgatattcgt | |
| | acctatccgatacagactgcgactcagctgacgagagaagtttatcttgatcttccgttcatcaacgagaatctgtctcctgca | |
| | gcagtttatccaaccttttctgccgctgaaagcgcaataattcgtagtccacatctggttgactttttaaattccttcacaatc | |
| | tataccgatagcctagcacggtcagcatactggggaggccacctggtgaatagttttcgtacgggcaccaccaccaatttgatt | |
| | cgttccccgttgtatggcagggaaggcaacacagaaaggccagttaccatcaccgcaagcccgagcgttcccatttttcgtact | |
| | ctgagctattttacgggtctggacaacagcaaccctgttgctgggattgagggtgttgagttccagaataccattagccgtagt | |
| | atctacagaaaaagcggacctatcgattcctttagcgaactgcccccgcaggatgcatctgtgagcccggccattggctatagc | |
| | caccgtctgtgccacgccacgtttctggagcgtatcagcgggccgcgaatcgctggtactgtgttttcctggacccatcgtagc | |
| | gccagtccgaccaatgaagttagcccttctcgtatcacgcaaatcccatgggtgaaggcacataccctcgcatcgggtgcatcc | |
| | gtcattaagggcccgggctttaccggtggggacattctgaccagaaactccatgggtgaactcggcaccctgcgcgtaacattt | |
| | actggtaggctgccccagagctactacattcgatttcgttatgccagcgtcgccaaccggagcggcacctttcgatattcccag | |
| | ccaccgtcctatggcatatcatttccaaaaaccatggacgcaggtgagcctctcactagccgttcgtttgcacataccaccctg | |
| | ttcacgccgattacgttttcccgtgcacaggaagaattcgacctctatatccagagtggcgtttacatcgaccgcatcgaattt | |
| | attccggttacggctacctttgaggcggagtatgatctagaaagagctcaaaaagctgtgaacgccctctttacctccaccaat | |
| | cagcttggcttgaaaactaacgttactgactatcacattgaccaagtgtccaacttggtcacctaccttagcgatgagttctgc | |
| | ctcgacgagaagcgtgaactctccgagaaagttaaacacgccaagcgtctcagcgacgagaggaatctcttgcaagactccaac | |
| | ttcaaagacatcaacaggcagccagaacgtggttggggtggaagcaccgggatcaccatccaaggaggcgacgatgtgttcaag | |
| | gagaactacgtcaccctctccggaactttcgacgagtgctaccctacctacttgtaccagaagatcgatgagtccaaactcaaa | |
| | gccttcaccaggtatcaacttagaggctacatcgaagacagccaagaccttgaaatctactcgatcaggtacaatgccaagcac | |
| | gagaccgtgaatgtcccaggtactggttccctctggccactttctgcccaatctcccattgggaagtgtggagagcctaacaga | |
| | tgcgctccacaccttgagtggaatcctgacttggactgctcctgcagggatggcgagaagtgtgcccaccattctcatcacttc | |
| | tccttggacatcgatgtgggatgtactgacctgaatgaggacctcggagtctgggtcatcttcaagatcaagacccaagacgga | |
| | cacgcaagacttggcaaccttgagtttctcgaagagaaaccattggtcggtgaagctctcgctcgtgtgaagagagcagagaag | |
| | aagtggagggacaaacgtgagaaactcgaatgggaaactaacatcgtttacaaggaggccaaagagtccgtggatgctttgttc | |
| | gtgaactcccaatatgatcagttgcaagccgacaccaacatcgccatgatccacgccgcagacaaacgtgtgcacagcattcgt | |
| | gaggcttacttgcctgagttgtccgtgatccctggtgtgaacgctgccatcttcgaggaacttgagggacgtatctttaccgca | |
| | ttctccttgtacgatgccagaaacgtcatcaagaacggtgacttcaacaatggcctcagctgctggaatgtgaaaggtcatgtg | |
| | gacgtggaggaacagaacaatcagcgttccgtcctggttgtgcctgagtgggaagctgaagtgtcccaagaggttagagtctgt | |
| | ccaggtagaggctacattctccgtgtgaccgcttacaaggagggatacggtgagggttgcgtgaccatccacgagatcgagaac | |
| | aacaccgacgagcttaagttctccaactgcgtcgaggaagaaatctatcccaacaacaccgttacttgcaacgactacactgtg | |
| | aatcaggaagagtacggaggtgcctacactagccgtaacagaggttacaacgaagctccttccgttcctgctgactatgcctcc | |
| | gtgtacgaggagaaatcctacacagatggcagacgtgagaacccttgcgagttcaacagaggttacagggactacacaccactt | |
| | ccagttggctatgttaccaaggagcttgagtactttcctgagaccgacaaagtgtggatcgagatcggtgaaaccgagggaacc | |
| | ttcatcgtggacagcgtggagcttctcttgatggaggaataa | |
| Primer 1 | gagtttttctgattaacagaagcttatggaaattaacaaccaga | SEQ |
| | | ID |
| | | NO: |
| | | 23 |
| Primer 2 | tgccaaatgtttgaacgatcggcgcgccttattcctccatcagcagca | SEQ |
| | | ID |
| | | NO: |
| | | 24 |
| Primer 3 | tgccaaatgtttgaacgatcggcgcgccttattcctccatgagcagtt | SEQ |
| | | ID |
| | | NO: |
| | | 25 |
| Primer 4 | tgccaaatgtttgaacgatcggcgcgccttattcctccatcaagagaa | SEQ |
| | | ID |
| | | NO: |
| | | 26 |
| Primer 5 | cagggtgtcacgttgcaaga | SEQ |
| | | ID |
| | | NO: |
| | | 27 |
| Primer 6 | ccgctcgtctggctaagatc | SEQ |
| | | ID |
| | | NO: |
| | | 28 |
| Probe 1 | tgcctgaaaccgaactgcccgctg | SEQ |
| | | ID |
| | | NO: |
| | | 29 |
| Primer 7 | gagggtgttgtggctggtattg | SEQ |
| | | ID |
| | | NO: |
| | | 30 |
| Primer 8 | tctcaactgtccaatcgtaagcg | SEQ |
| | | ID |
| | | NO: |
| | | 31 |
| Probe 2 | cttacgctgggccctggaaggctag | SEQ |
| | | ID |
| | | NO: |
| | | 32 |
| The amino | MEINNQNQCVPYNCLSNPKEIILGEERLETGNTVADISLGLINFLYSNFVPGGGFI | SEQ |
| acid | VGLLELIWGFIGPSQWDIFLAQIEQLISQRIEEFARNQAISRLEGLSNLYKVYVRA | ID |
| sequence | FSDWEKDPTNPALREEMRIQFNDMNSALITAIPLFRVQNYEVALLSVYVQAANL | NO: |
| of BD1- | HLSILRDVSVFGERWGYDTATINNRYSDLTSLIHVYTNHCVDTYNQGLRRLEGR | 33 |
| 022S | FLSDWIVYNRFRRQLTISVLDIVAFFPNYDIRTYPIQTATQLTREVYLDLPFINEN | |
| | LSPAAVYPTFSAAESAIIRSPHLVDFLNSFTIYTDSLARSAYWGGHLVNSFRTGT | |
| | TTNLIRSPLYGREGNTERPVTITASPSVPIFRTLSYFTGLDNSNPVAGIEGVEFQN | |
| | TISRSIYRKSGPIDSFSELPPQDASVSPAIGYSHRLCHATFLERISGPRIAGTVFSW | |
| | THRSASPTNEVSPSRITQIPWVKAHTLASGASVIKGPGFTGGDILTRNSMGELGT | |
| | LRVTFTGRLPQSYYIRFRYASVANRSGTFRYSQPPSYGISFPKTMDAGEPLTSRS | |
| | FAHTTLFTPITFSRAQEEFDLYIQSGVYIDRIEFIPVTATFEAEYDLERAQKAVNA | |
| | LFTST | |
| The | atggaaattaacaaccagaatcaatgtgtcccgtacaactgtttaagcaatcctaaggagataattctgggtgaagagcggcta | SEQ |
| nucleo- | gaaaccgggaacaccgttgctgatattagtctggggctgattaattttttgtattctaattttgtgccgggggcggttttatag | ID |
| tide | taggcctgctagagcttatctggggatttatcggtccgtcccagtgggatatctttctggcccagattgagcagctgatttcac | NO: |
| sequence | agaggattgaagaatttgctagaaaccaagcaatttccaggctcgaagggttatctaatttgtataaagtatatgtacgggctt | 34 |
| encoding | ttagcgactgggagaaagatccgaccaatccagcactgcgtgaagagatgcggattcagttcaatgacatgaacagcgcattga | |
| BD1-022S | tcaccgcaattccgctcttccgcgtacaaaattatgaagtggcgttgctgtccgtctatgttcaagccgctaacttacacctaa | |
| | gcattctgcgggatgtgagtgtttttggcgaacgatggggttatgataccgcgacgatcaataatagatattccgatctgacca | |
| | gcctcatccatgtttatacaaaccattgtgttgatacgtataaccagggactcagacgtctggagggtcggttcttatccgatt | |
| | ggatcgtatataaccgttttagacgccagttgacaattagcgttctggatattgtggcgttcttcccaaattatgatattcgta | |
| | cctatccgatacagactgcgactcagctgacgagagaagtttatcttgatcttccgttcatcaacgagaatctgtctcctgcag | |
| | cagtttatccaaccttttctgccgctgaaagcgcaataattcgtagtccacatctggttgactttttaaattccttcacaatct | |
| | ataccgatagcctagcacggtcagcatactggggaggccacctggtgaatagttttcgtacgggcaccaccaccaatttgattc | |
| | gttccccgttgtatggcagggaaggcaacacagaaaggccagttaccatcaccgcaagcccgagcgttcccatttttcgtactc | |
| | tgagctattttacgggtctggacaacagcaaccctgttgctgggattgagggtgttgagttccagaataccattagccgtagta | |
| | tctacagaaaaagcggacctatcgattcctttagcgaactgcccccgcaggatgcatctgtgagcccggccattggctatagcc | |
| | accgtctgtgccacgccacgtttctggagcgtatcagcgggccgcgaatcgctggtactgtgttttcctggacccatcgtagcg | |
| | ccagtccgaccaatgaagttagcccttctcgtatcacgcaaatcccatgggtgaaggcacataccctcgcatcgggtgcatccg | |
| | tcattaagggcccgggctttaccggtggggacattctgaccagaaactccatgggtgaactcggcaccctgcgcgtaacattta | |
| | ctggtaggctgccccagagctactacattcgatttcgttatgccagcgtcgccaaccggagcggcacctttcgatattcccagc | |
| | caccgtcctatggcatatcatttccaaaaaccatggacgcaggtgagcctctcactagccgttcgtttgcacataccaccctgt | |
| | tcacgccgattacgttttcccgtgcacaggaagaattcgacctctatatccagagtggcgtttacatcgaccgcatcgaattta | |
| | ttccggttacggctacctttgaggcggagtatgatctagaaagagctcaaaaagctgtgaacgccctctttacctccacctaa | |
| Primer 9 | tgccaaatgtttgaacgatcggcgcgccttaggtggaggtaaaga | SEQ |
| | | ID |
| | | NO: |
| | | 35 |

The technical solution of the present invention will be further illustrated through the following specific Examples.

### Example 1: Mutagenesis Modification of the Cry1Da1 Protein

Through the analysis of protein structure, we identified that the loop region of Domain II region of the Cry1Da1 protein (referred to as BD1-001 herein) may play an important role in receptor binding. We focused on modifying this region by mutating multiple amino acid sites. In addition, we identified that the substitutions in the C-terminal increased the insect-resistant activity of the protein to some extent. The modification manners of the proteins are listed in Table 1.

TABLE 1
The modified Cry1Da1 proteins and the modification manners thereof

| Protein number | Modification manners |
|---|---|
| BD1-011 | Cry1Da1 + S282L_Y316S_I368P |
| BD1-013 | Cry1Da1 + S282V_Y316S_I368F |
| BD1-014 | Cry1Da1 + S282L + Y316S + I368F |
| BD1-015 | Cry1Da1 + S282V + Y316T + I368P |
| BD1-018 | Cry1Da1 + S282V_Y316S_I368F + C-terminal of Cry8Ea1 |
| BD1-019 | Cry1Da1 + S282V_Y316S_I368F_F595L_D600N + C-terminal of Cry8Ea1 |
| BD1-020 | Cry1Da1 + S282V_Y316S_I368Y + C-terminal of Cry8Ea1 |
| BD1-021 | Cry1Da1 + S282V_Y316S_I368W + C-terminal of Cry8Ea1 |
| BD1-022 | Cry1Da1 + S282V_Y316S_I368F + C-terminal of Cry1Ac |
| BD1-022S | Cry1Da1 + S282V_Y316S_I368F + Truncated C-terminal of Cry1Ac |

In the modification manners, the first amino acid abbreviation represents the original amino acid in the protein, the following number represents the position of the amino acid, and the second amino acid abbreviation represents the amino acid at that position in the modified protein. The C-terminal of Cry8Ea1 and C-terminal of Cry1Ac refer to replacing the amino acid sequence after position 606 of the original amino acid sequence with the corresponding amino acid sequences of Cry8Ea1 and Cry1Ac proteins, respectively.

### Example 2: Construction of In Vitro Expression Vectors of the Cry1Da1 Protein and Protein Purification

### 1. Construction of the Recombinant Cloning Vectors Containing Cry1Da1-Like Genes

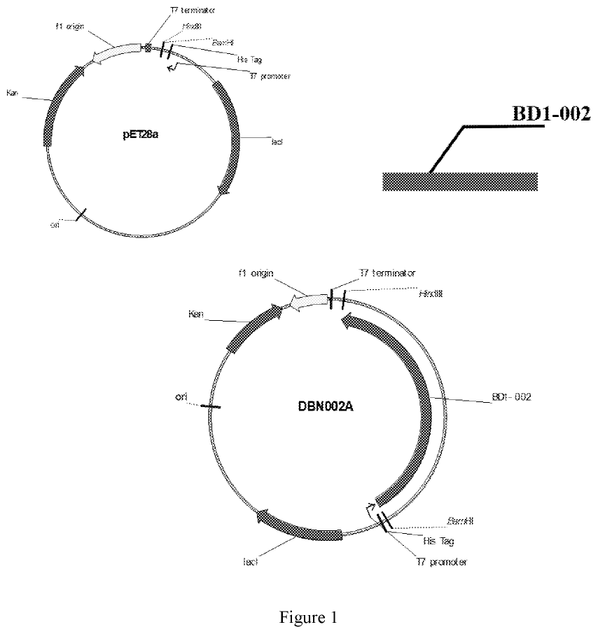

1) After site-specific mutagenesis modification of the amino acid positions in the amino acid sequence BD1-001, Cry1Da1-like amino acid sequences BD1-011, BD1-013, BD1-014, BD1-015, BD1-018, BD1-019, BD1-020, BD1-021, and BD1-022 were obtained, wherein the amino acid sequence BD1-022S were obtained by truncating the C-terminal of the amino acid sequence BD1-022.
2) Taking the gene of the control protein BD1-002 as an example, by using the restriction sites of BamHI and Hind III, the nucleotide sequence of BD1-002 (SEQ ID NO: 13) was cloned into the plasmid pET28a (Novagen, USA, CAT: 69864-3) to obtain the recombinant vector DBN002A. The construction process is shown in FIG. 1 (wherein f1 origin represents the origin of replication of phage f1; Kan represents the kanamycin resistance gene; T7 promoter represents the promoter of T7 RNA polymerase; His Tag represents the His tag; BD1-002 represents the nucleotide sequence of BD1-002 (SEQ ID NO: 13); T7 terminator represents the T7 terminator). E. coli BL21 (DE3) competent cells (Transgen, China, CAT: CD501) were transformed through heat shock method. The heat shock conditions were as follows: 50 μL of E. coli BL21 (DE3) competent cells and 10 μL of plasmid DNA were incubated in water bath at 42° C. for 30s; cultured by shaking (100 rpm) at 37° C. for 1h; then the cultured products were plated on a LB solid plate (1% Tryptone, 1% NaCl, 0.5% yeast extract, and 1.5% agar) containing 50 mg/L Kanamycin and cultured at 37° C. for 12 h. The monoclonal colonies were picked up and inoculated into 5 mL LB liquid medium (1% Tryptone, 1% NaCl, 0.5% yeast extract, pH adjusted to 7.5 with NaOH) supplemented with kanamycin at a final concentration of 50 mg/L, and cultured on a shaking incubator (200 rpm) at 37° C. for 16 h. The plasmid was extracted using the AxyPrep plasmid DNA extraction kit (CORNING, China, CAT: AP-MN-P-50). The resultant plasmids were verified by enzymatic digestion with BamHI and HindIII, and the positive clones were verified through sequencing. The results showed that the target nucleotide sequence inserted into the positive recombinant cloning vector was the nucleotide sequence as shown in SEQ ID NO. 13 in the sequence listing; that is, the nucleotide sequence of BD1-002 was correctly inserted.
3) Following the above process for constructing a recombinant cloning vector, the nucleotide sequence of BD1-011 was ligated into the plasmid pET28a to obtain the recombinant cloning vector DBN011A, wherein BD1-011 was the nucleotide sequence of BD1-011 (SEQ ID NO: 14). Enzymatic digestion and sequencing verification result showed that the nucleotide sequence of BD1-011 in the recombinant cloning vector DBN011A was correctly inserted.
4) Following the above process for constructing a recombinant cloning vector, the nucleotide sequence of BD1-013 was ligated into the plasmid pET28a to obtain the recombinant cloning vector DBN013A, wherein BD1-013 was the nucleotide sequence of BD1-013 (SEQ ID NO: 15). Enzymatic digestion and sequencing verification result showed that the nucleotide sequence of BD1-013 in the recombinant cloning vector DBN013A was correctly inserted.
5) Following the above process for constructing a recombinant cloning vector, the nucleotide sequence of BD1-014 was ligated into the plasmid pET28a to obtain the recombinant cloning vector DBN014A, wherein BD1-014 was the nucleotide sequence of BD1-014 (SEQ ID NO: 16). Enzymatic digestion and sequencing verification result showed that the nucleotide sequence of BD1-014 in the recombinant cloning vector DBN014A was correctly inserted.
6) Following the above process for constructing a recombinant cloning vector, the nucleotide sequence of BD1-015 was ligated into the plasmid pET28a to obtain the recombinant cloning vector DBN015A, wherein BD1-015 was the nucleotide sequence of BD1-015 (SEQ ID NO: 17). Enzymatic digestion and sequencing verification result showed that the nucleotide sequence of BD1-015 in the recombinant cloning vector DBN015A was correctly inserted.
7) Following the above process for constructing a recombinant cloning vector, the nucleotide sequence of BD1-018 was ligated into the plasmid pET28a to obtain the recombinant cloning vector DBN018A, wherein BD1-018 was the nucleotide sequence of BD1-018 (SEQ ID NO: 18). Enzymatic digestion and sequencing verification result showed that the nucleotide sequence of BD1-018 in the recombinant cloning vector DBN018A was correctly inserted.
8) Following the above process for constructing a recombinant cloning vector, the nucleotide sequence of BD1-019 was ligated into the plasmid pET28a to obtain the recombinant cloning vector DBN019A, wherein BD1-019 was the nucleotide sequence of BD1-019 (SEQ ID NO: 19). Enzymatic digestion and sequencing verification result showed that the nucleotide sequence of BD1-019 in the recombinant cloning vector DBN019A was correctly inserted.
9) Following the above process for constructing a recombinant cloning vector, the nucleotide sequence of BD1-020 was ligated into the plasmid pET28a to obtain the recombinant cloning vector DBN020A, wherein BD1-020 was the nucleotide sequence of BD1-020 (SEQ ID NO: 20). Enzymatic digestion and sequencing verification result showed that the nucleotide sequence of BD1-020 in the recombinant cloning vector DBN020A was correctly inserted.
10) Following the above process for constructing a recombinant cloning vector, the nucleotide sequence of BD1-021 was ligated into the plasmid pET28a to obtain the recombinant cloning vector DBN021A, wherein BD1-021 was the nucleotide sequence of BD1-021 (SEQ ID NO: 21). Enzymatic digestion and sequencing verification result showed that the nucleotide sequence of BD1-021 in the recombinant cloning vector DBN021A was correctly inserted.
11) Following the above process for constructing a recombinant cloning vector, the nucleotide sequence of BD1-022 was ligated into the plasmid pET28a to obtain the recombinant cloning vector DBN022A, wherein BD1-022 was the nucleotide sequence of BD1-022 (SEQ ID NO: 22). Enzymatic digestion and sequencing verification result showed that the nucleotide sequence of BD1-022 in the recombinant cloning vector DBN022A was correctly inserted.
12) Following the above process for constructing a recombinant cloning vector, the nucleotide sequence of BD1-022S was ligated into the plasmid pET28a to obtain the recombinant cloning vector DBN022SA, wherein BD1-022S was the nucleotide sequence of BD1-022S (SEQ ID NO: 34). Enzymatic digestion and sequencing verification result showed that the nucleotide sequence of BD1-022S in the recombinant cloning vector DBN022SA was correctly inserted.

### 2. In Vitro Expression of Cry1Da1-Like Proteins

1) Positive single colonies were picked up and inoculated into 5 mL LB liquid medium supplemented with kanamycin at a final concentration of 50 mg/L, and cultured on a shaking incubator (220 rpm) at 37° C. for 16 h to obtain activated strains.
2) The bacterial culture solution was transferred to 2×YT medium (1.6% Tryptone, 0.5% NaCl, 1% yeast extract) at a ratio of 1:10, cultured on a shaking incubator (220 rpm) at 37° C. for 1h.
3) When the OD600 of the culture solution was 0.6~0.8, it was added with IPTG at a final concentration of 0.5 mM to induce expression and cultured on a shaking incubator (220 rpm) at 37° C. for 6h.
4) The bacterial cells were harvested by centrifugation at 7000 rpm for 5 min and the supernatant was discarded. The bacterial cells were resuspended with an appropriate amount of PBS buffer and sonicated to obtain lysis bacterial solution. The lysed bacterial solution was centrifuged at 7000 rpm for 5 min to obtain soluble fraction and insoluble fraction, wherein the insoluble fraction was resuspended with PBS buffer.
5) An appropriate amount of samples was taken for SDS-PAGE detection. The results showed that the target protein was mainly present in the soluble fraction.

### 3. Purification of Cry1Da1-Like Proteins

1) The soluble fraction was purified by the AKTA rapid purification system with a HisTrap HP nickel column to obtain purified Cry1Da1-like proteins. The purified proteins were desalted and purified by a HiTrap Desalting column. The operation procedures refer to the AKTA operation manual.
2) An appropriate amount of the desalted and purified samples was taken for SDS-PAGE detection.
3) The protein concentration of the desalted protein solution was calculated using a BSA standard curve.
4) The purified proteins ware stored at −20° C. for further use.

### Example 3: Resistance Test of the Modified Protein Against Lepidoptera Insects

3.1 Feed Bioassay of Spodoptera frugiperda

The prokaryotically-expressed modified proteins were fed to Spodoptera frugiperda to compare their insect-resistant activities. The concentrated protein solution was mixed well with the feed of Spodoptera frugiperda (final concentration: 1 μg/g) and the mixture was placed in Petri dishes. Healthy and unfed newly hatched larvae of Spodoptera frugiperda were selected as the test insects. Ten larvae were introduced into a Petri dish, and the Petri dishes for insect test were covered and placed under the following conditions: 25-28° C., relative humidity 70% and a photoperiod (light/dark) of 16:8 hours until the experiment finished on the third day. By comparing mortality and inhibition rate of the test insects, the insect-resistant activity of each protein was divided into four grades which were denoted by “+”, wherein more “+” represents better insect-resistant effect. The results are shown in Table 2.

TABLE 2
Comparison of the insect-resistant activities of the
modified proteins against Spodoptera frugiperda

| Protein number | Insect-resistant effect |
|---|---|
| BD1-002 | +++ |
| BD1-011 | + |
| BD1-013 | +++ |
| BD1-014 | ++ |
| BD1-015 | ++ |
| BD1-018 | ++++ |
| BD1-019 | ++++ |
| BD1-020 | ++ |
| BD1-021 | ++++ |
| BD1-022 | ++++ |
| BD1-022S | ++++ |

more “+” represents better insect-resistant effect.

The modified proteins with better resistance against Spodoptera frugiperda were selected, and their insect-resistant activities were compared with the patented protein BD1-002 (the sequence of BD1-002 (Cry1Da1_7) is a patent-protected sequence modified based on BD1-001 (Cry1Da1), and the sequences of BD1-001 and BD1-002 are from the public information of patent U.S. Pat. No. 10,287,605B2 or NCBI GenBank: CAA38099.1, which is a full-length amino acid sequence of 1165 aa). Using the same protein feeding method as described above, the final protein concentration in the feed was adjusted to 1 μg/g. After 3 days of treatment, the mortality of Spodoptera frugiperda larvae was calculated and collected: mortality=(the number of dead larvae/total number of introduced larvae)×100%. The feed supplemented with only CBS buffer and the feed supplemented with only sterile water were taken as negative controls. The mortality of the CBS buffer treatment group was taken as the control mortality, and the corrected mortality was calculated based on the control mortality: corrected mortality=(the treatment mortality−the control mortality)/(1−the control mortality)×100%. Each system involved six replicates and the experiment was performed twice. The results are shown in Table 3.

TABLE 3
The insect-resistant results of feeding Spodoptera frugiperda
with the proteins (3 days)

| Protein number | Corrected mortality % |
|---|---|
| BD1-002 | 21.4 ± 8.92 |
| BD1-018 | 63.7 ± 5.13 * |
| BD1-019 | 61.4 ± 8.57 * |
| BD1-021 | 59.0 ± 11.0 * |
| BD1-022 | 65.3 ± 9.65 * |
| BD1-022S | 65.7 ± 6.11 * |

* indicates that the data show a significant difference.

From Table 3, it can be seen that as compared with the BD1-002 protein, the mortality of Spodoptera frugiperda fed with the above modified proteins was significantly increased, indicating that the insect-resistant activities of the above modified proteins in the present application against Spodoptera frugiperda were significantly better than that of the patented protein BD1-002.

3.2 Feed Bioassay of Helicoverpa armigera

The concentrated protein solution was mixed well with the feed of Helicoverpa armigera (final concentration: 20 μg/g) and the mixture was placed in Petri dishes. Healthy and unfed newly hatched larvae of Helicoverpa armigera were selected as the test insects. One larva was introduced into each well and twelve larvae constitute one replicate. The Petri dishes were covered and placed under the following conditions: 25-28° C., relative humidity 70% and a photoperiod (light/dark) of 16:8 hours until the experiment finished on the seventh day. The mortality of Helicoverpa armigera larvae was calculated and collected: mortality=(the number of dead larvae/total number of introduced larvae)×100%. The feed supplemented with only CBS buffer and the feed supplemented with only sterile water were taken as negative controls. The mortality of the CBS buffer treatment group was taken as the control mortality, and the corrected mortality was calculated based on the control mortality: corrected mortality=(the treatment mortality−the control mortality)/(1−the control mortality)×100%. Each system involved six replicates and the experiment was performed twice. The results are shown in Table 4.

TABLE 4
The insect-resistant results of feeding Helicoverpa armigera
with the proteins (7 days)

| Protein number | Corrected mortality % |
|---|---|
| BD1-002 | 6.5 ± 5.59 |
| BD1-018 | 52.0 ± 7.21 * |
| BD1-019 | 54.8 ± 5.59 * |
| BD1-021 | 50.7 ± 8.08 * |
| BD1-022 | 50.3 ± 9.65 * |
| BD1-022S | 50.0 ± 9.54 * |

* indicates that the data show a significant difference.

From Table 4, it can be seen that as compared with the BD1-002 protein, the mortality of Helicoverpa armigera fed with the above modified proteins of the present application was significantly increased, indicating that the insect-resistant activity of the modified proteins in the present application against Helicoverpa armigera was significantly better than that of the patented protein BD1-002.

3.3 Feed Bioassay of Argyrogramma agnata

The concentrated protein solution was mixed well with the feed of Argyrogramma agnata (final concentration: 100 μg/g) and the mixture was placed in Petri dishes. Healthy and unfed newly hatched larvae of Argyrogramma agnata were selected as the test insects, 10 larvae were introduced into a Petri dish, and the Petri dishes were covered and placed under the following conditions: 25-28° C., relative humidity 70% and a photoperiod (light/dark) of 16:8 hours until the experiment finished on the third day. The mortality of Argyrogramma agnata larvae was calculated and collected: mortality=(the number of dead larvae/total number of introduced larvae)×100%. The feed supplemented with only CBS buffer and the feed supplemented with only sterile water were taken as negative controls. The mortality of the CBS buffer treatment group was taken as the control mortality, and the corrected mortality was calculated based on the control mortality: corrected mortality=(the treatment mortality−the control mortality)/(1−the control mortality)×100%. Each system involved six replicates and the experiment was performed twice. The results are shown in Table 5.

TABLE 5
The insect-resistant results of feeding Argyrogramma agnata
with the protein (3 days)

| Protein number | Corrected mortality % |
|---|---|
| BD1-002 | 42 ± 16 |
| BD1-018 | 63 ± 14 |
| BD1-019 | 70 ± 21 * |
| BD1-021 | 60 ± 16 |
| BD1-022 | 61 ± 24 |
| BD1-022S | 65 ± 8 |

* indicates that the data show a significant difference.

From Table 5, it can be seen that as compared with the BD1-002 protein, the mortality of Argyrogramma agnata fed with the modified proteins of the present application was significantly increased, indicating that the insect-resistant activities of the modified proteins in the present application against Argyrogramma agnata was significantly better than that of the patented protein BD1-002.

### Example 4: Construction of Plant Transformants Encoding Engineered Proteins

### 4.1 Construction of Transgenic Corn Plants

### 4.1.1 Construction of Expression Cassettes

### 4.1.1.1 Construction of the Intermediate Vector Containing the Target Gene

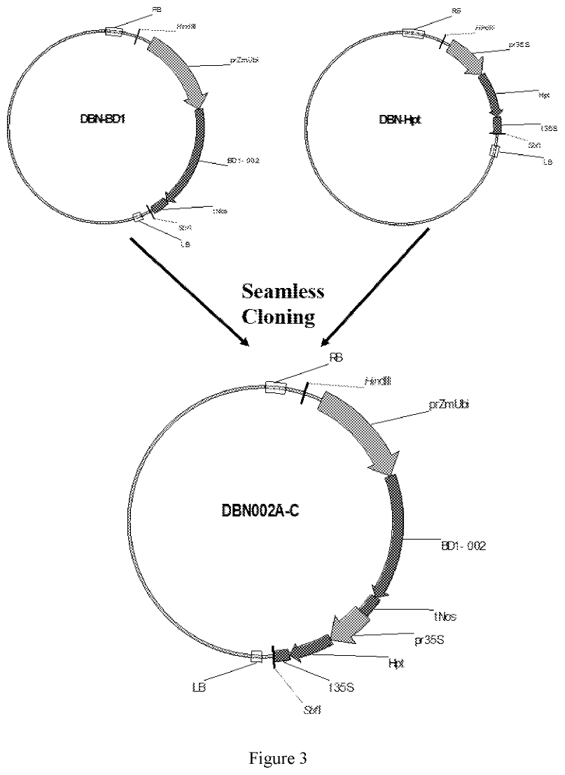

The promoter was the maize ubiquitin gene promoter prZmUbi, and the terminator was the nopaline synthase (nos) terminator tNos. The detailed construction process is as follows:
1) The plasmid pCAMBIA2301 was used as the template to amplify the promoter pZmUbi by PCR. The promoter pZmUbi was added with a 20 bp homologous arm corresponding to the HindIII restriction site and its upstream region in the DBN-backbone at its 5′ end and with a 20 bp homologous arm corresponding to the 5′ end of the BD1-002 gene at its 3′ end to obtain the prZmUbi promoter fragment.
2) The plasmid DBN002A was used as the template to amplify the target gene BD1-002 by PCR. The target gene BD1-002 was added with a 20 bp homologous arm corresponding to the 3′ end of the promoter prZmUbi at its 5′ end and with a 20 bp homologous arm corresponding to the 5′ end of the tNos gene at its 3′ end to obtain the target gene BD1-002 fragment.
3) The plasmid pCAMBIA2301 was used as the template to amplify the terminator tNos by PCR. The terminator tNos was added with a 20 bp homologous arm corresponding to the 3′ end of the BD1-002 gene at its 5′ end and with a 20 bp homologous arm corresponding to the SbfI restriction site and its downstream region in the DBN-backbone at its 3′ end to obtain the tNos terminator fragment.
4) The DBN-backbone was double-digested with the enzymes HindIII and SbfI to obtain the HindIII-DBN-backbone-SbfI fragment.
5) The four fragments obtained in the above steps were ligated and transformed by seamless cloning to generate the intermediate vector DBN-BD1 (RB: right border; prZmUbi: corn ubiquitin gene promoter; BD1-002: the nucleotide sequence of BD1-002 (SEQ ID NO: 13); tNos: nopaline synthase (nos) terminator; LB: left border), as shown in FIG. 3.

### 4.1.1.2 Construction of the Intermediate Vector Containing the Reporter Gene Hpt

The reporter gene Hpt is the hygromycin phosphotransferase gene, the promoter is the cauliflower mosaic virus 35S promoter pr35S, and the terminator is the cauliflower mosaic virus 35S terminator t35S. The detailed construction process is as follows:
1) The plasmid pCAMBIA2301 was used as the template to amplify the promoter pr35S-06 by PCR. The promoter pr35S-06 was added with a 20 bp homologous arm corresponding to the HindIII restriction site and its upstream region in the DBN-backbone at its 5′ end and with a 20 bp homologous arm corresponding to the 5′ end of the Hpt gene at its 3′ end to obtain the pr35S promoter fragment.
2) The plasmid pCAMBIA2301 was used as the template to amplify the reporter gene Hpt by PCR. The reporter gene Hpt was added with a 20 bp homologous arm corresponding to the 3′ end of the promoter pr35S at its 5′ end and with a 20 bp homologous arm corresponding to the 5′ end of the terminator t35S at its 3′ end to obtain the gene Hpt fragment.
3) The plasmid pCAMBIA2301 was used as the template to amplify the terminator t35S by PCR. The terminator t35S was added with a 20 bp homologous arm corresponding to the 3′ end of the Hpt gene at its 5′ end and with a 20 bp homologous arm corresponding to the SbfI restriction site and its downstream region in the DBN-backbone at its 3′ end to obtain the t35S terminator fragment.
4) The DBN-backbone was double-digested with the enzymes HindIII and SbfI to obtain the HindIII-DBN-backbone-SbfI fragment.
5) The four fragments obtained in the above steps were ligated and transformed by seamless cloning to generate the intermediate vector pDBN-Hpt (RB: right border; pr35S: cauliflower mosaic virus 35S promoter; Hpt: hygromycin phosphotransferase gene; t35S: cauliflower mosaic virus 35S terminator; LB: left border), as shown in FIG. 3.

### 4.1.1.3 Construction of the Final Vector

1) The DBN-backbone was double-digested with the enzymes HindIII and SbfI to obtain the HindIII-DBN-backbone-SbfI fragment.
2) The plasmid pDBN-BD1 was used as the template to amplify the BD1-002 cassette by PCR. The BD1-002 cassette was added with a 20 bp homologous arm corresponding to the HindIII restriction site and its upstream region in the DBN-backbone at its 5′ end and with a 20 bp homologous arm corresponding to the 5′ end of the Hpt cassette at its 3′ end to obtain the BD1-002 cassette fragment.
3) The plasmid pDBN-Hpt was used as the template to amplify the Hpt cassette. The Hpt cassette was added with a 20 bp homologous arm corresponding to the 3′ end of the BD1-002 cassette at its 5′ end and with a 20 bp homologous arm corresponding to the SbfI restriction site and its downstream region in the DBN-backbone at its 3′ end to obtain the Hpt cassette fragment.
4) The three fragments obtained in the above steps were ligated and transformed by seamless cloning to generate the final vector DBN002A-C(RB: right border; prZmUbi: corn ubiquitin gene promoter; BD1-002: the nucleotide sequence of BD1-002 (SEQ ID NO: 13); tNos: nopaline synthase (nos) terminator; pr35S: cauliflower mosaic virus 35S promoter; Hpt: hygromycin phosphotransferase gene; t35S: cauliflower mosaic virus 35S terminator; LB: left border), as shown in FIG. 3.

### 4.1.1.4 Transformation and Identification of the Vector

The recombinant expression vector DBN002A-C was transformed into E. coli T1 competent cells through heat shock method. The heat shock conditions were as follows: 50 μL of E. coli T1 competent cells and 10 μL of plasmid DNA were incubated in water bath at 42° C. for 30s; cultured by shaking at 37° C. for 1h (shaking at 100 rpm on a shaking incubator); then the cultured products were plated on a LB solid plate containing 50 mg/L Kanamycin and cultured at 37° C. for 12 h. The single colonies were picked out and inoculated into 5 mL LB liquid medium supplemented with kanamycin at a final concentration of 50 mg/L, and cultured on a shaking incubator (200 rpm) at 37° C. for 16 h. The single colonies were picked up and inoculated into LB liquid medium supplemented with kanamycin at a final concentration of 50 mg/L, and cultured at 37° C. overnight. The plasmid was extracted using the AxyPrep plasmid DNA extraction kit. The extracted plasmids were identified by digestion with the restriction enzymes SbfI and HindIII, and the positive clones were identified through sequencing. The results showed that the nucleotide sequence between the SbfI and HindIII restriction sites in the recombinant expression vector DBN002A-C contained the nucleotide sequence as shown in SEQ ID NO: 13 in the sequence listing, i.e., the nucleotide sequence of BD1-002.

Following the above process for constructing DBN002A-C, the recombinant expression vector DBN018A-C was obtained. Enzymatic digestion and sequencing verified that the nucleotide sequence in the recombinant expression vector DBN018A-C contained the nucleotide sequence as shown in SEQ ID NO: 18 in the sequence listing, i.e., the nucleotide sequence of BD1-018.

Following the above process for constructing DBN002A-C, the recombinant expression vector DBN019A-C was obtained. Enzymatic digestion and sequencing verified that the nucleotide sequence in the recombinant expression vector DBN019A-C contained the nucleotide sequence as shown in SEQ ID NO: 19 in the sequence listing, i.e., the nucleotide sequence of BD1-019.

Following the above process for constructing DBN002A-C, the recombinant expression vector DBN021A-C was obtained. Enzymatic digestion and sequencing verified that the nucleotide sequence in the recombinant expression vector DBN021A-C contained the nucleotide sequence as shown in SEQ ID NO: 21 in the sequence listing, i.e., the nucleotide sequence of BD1-021.

Following the above process for constructing DBN002A-C, the recombinant expression vector DBN022A-C was obtained. Enzymatic digestion and sequencing verified that the nucleotide sequence in the recombinant expression vector DBN022A-C contained the nucleotide sequence as shown in SEQ ID NO: 22 in the sequence listing, i.e., the nucleotide sequence of BD1-022.

Following the above process for constructing DBN002A-C, the recombinant expression vector DBN022SA-C was obtained. Enzymatic digestion and sequencing verified that the nucleotide sequence in the recombinant expression vector DBN022SA-C contained the nucleotide sequence as shown in SEQ ID NO: 34 in the sequence listing, i.e., the nucleotide sequence of BD1-022S.

4.1.2 Transformation of Agrobacterium with the Recombinant Expression Vectors

The correctly constructed recombinant expression vectors DBN002A-C, DBN018A-C, DBN019A-C, DBN021A-C, DBN022A-C, and DBN022SA-C were transformed into Agrobacterium tumefaciens strain LBA4404 (InVitrogen, Chicago, USA, CAT: 18313-015) using the liquid nitrogen method. The transformation conditions were as follows: 100 μL Agrobacterium tumefaciens strain LBA4404 and 3 μL plasmid DNA (recombinant expression vector) were placed into liquid nitrogen for 10 min and then incubated in water bath at 37° C. for 10 min. The transformed Agrobacterium tumefaciens strain LBA4404 was inoculated into an LB tube and cultured by shaking (200 rpm) at 28° C. for 2 hours, and then plated on a LB plate containing 50 mg/L of Rifampicin and 100 mg/L of Kanamycin until positive single colonies appeared. The single colonies were picked up and cultured, and the plasmids thereof were extracted. The recombinant expression vectors DBN002A-C, DBN018A-C, DBN019A-C, DBN021A-C, DBN022A-C, and DBN022SA-C were verified by enzymatic digestion with restriction enzymes. The results showed that the structures of the recombinant expression vectors DBN002A-C, DBN018A-C, DBN019A-C, DBN021A-C, DBN022A-C, and DBN022SA-C were correct.

### 4.1.3 Agrobacterium-Mediated Transformation of Corn Plants

According to the conventional Agrobacterium-mediated transformation method, the sterilely cultured young embryos of the corn cultivar TJ806 were co-cultured with the Agrobacterium strains transformed with the recombinant expression vectors so as to transform the T-DNA into the recombinant expression vector DBN002A-C into the corn genome and then obtain the corn plants into which the nucleotide sequence of BD1-002 was transformed, with wild type corn plants as control.

Following the above method for obtaining the corn plants containing the BD1-002, the corn plants into which the nucleotide sequences of the BD1-018, BD1-019, BD1-021, BD1-022, and BD1-022S were transformed were obtained.

As to Agrobacterium-mediated transformation of corn plants, in brief, immature young embryos of corn were isolated and contacted with Agrobacterium suspension, wherein the Agrobacterium could deliver the nucleotide sequence of Cry1Da1 into at least one cell in one of the young embryos (Step 1: infection step). In this step, preferably, the young embryo was immersed in the Agrobacterium suspension (OD₆₆₀=0.4~0.6, infection medium (4.3 g/L of MS salt, MS vitamins, 300 mg/L of casein, 68.5 g/L of sucrose, 36 g/L of glucose, 40 mg/L of acetosyringone (AS), 1 mg/L of 2,4-dichlorophenoxyacetic acid (2,4-D), pH=5.3)) to initiate the inoculation. The young embryos and Agrobacterium were co-cultured for 3 days (Step 2: co-culture step). Preferably, after the infection step, the young embryos were cultured on solid medium (4.3 g/L of MS salt, MS vitamins, 300 mg/L of casein, 20 g/L of sucrose, 10 g/L of glucose, 100 mg/L of acetosyringone (AS), 1 mg/L of 2,4-dichlorophenoxyacetic acid (2,4-D), and 8 g/L of agar, pH=5.8). After this co-culture stage, an optional “recovery” step could be performed. In the “recovery” step, the recovery medium (4.3 g/L of MS salt, MS vitamins, 300 mg/L of casein, 30 g/L of sucrose, 1 mg/L of 2,4-dichlorophenoxyacetic acid (2,4-D), and 8 g/L of agar, pH=5.8) contained at least one antibiotic (cephamycin) known to inhibit Agrobacterium growth, but no selective agent for plant transformants (Step 3: recovery step). The young embryos were cultured on solid medium containing antibiotics but no selective agents so as to eliminate Agrobacterium and to provide a recovery period for the infected cells to generate callus. Then, the callus was inoculated and cultured on a medium containing a selective agent (hygromycin) and the growing transformed callus was selected (Step 4: selection step). Preferably, the callus was cultured on selective solid medium containing a selective agent (4.3 g/L of MS salt, MS vitamins, 300 mg/L of casein, 5 g/L of sucrose, 50 mg/L of hygromycin, 1 mg/L of 2,4-dichlorophenoxyacetic acid (2,4-D), and 8 g/L of agar, pH-5.8), resulting in selective growth of the transformed cells. Then, the callus was regenerated into plants (Step 5: regeneration step). Preferably, the callus growing on medium containing a selective agent was cultured on solid medium (MS differentiation medium and MS rooting medium) to regenerate plants. The resistant callus obtained by selection was transferred to the MS differentiation medium (4.3 g/L MS salt, MS vitamins, 300 mg/L of casein, 30 g/L of sucrose, 2 mg/L of 6-benzyladenine, 50 mg/L of hygromycin, and 8 g/L of agar, pH=5.8) and cultured at 25° C. for differentiation. The differentiated seedlings were transferred to the MS rooting medium (2.15 g/L of MS salt, MS vitamins, 300 mg/L of casein, 30 g/L of sucrose, 1 mg/L indole-3-acetic acid, and 8 g/L of agar, pH=5.8) and cultured at 25° C. until they reached about 10 cm in height, and then, they were moved to a greenhouse and cultured until fructification. In the greenhouse, the plants were cultured at 28° C. for 16 hours and at 20° C. for 8 hours every day.

### 4.1.4 Identification of Transgenic Corn Materials

About 100 mg of leaves from corn plants transformed with the nucleotide sequences of BD1-002, BD1-018, BD1-019, BD1-021, BD1-022, and BD1-022S were respectively taken as samples. Genomic DNA was extracted using the DNeasy Plant Maxi Kit from Qiagen and the copy numbers of the Hpt gene were detected using the Taqman probe-based fluorescence quantitative PCR assay to determine the copy numbers of the Cry1Da1 gene. Wild type TJ806 corn plants were taken as controls. The analysis was performed according to the above process. The experiment was carried out three times. The analysis of the experimental results of the copy number of the Hpt reporter gene showed that the nucleotide sequences of BD1-002, BD1-018, BD1-019, BD1-021, BD1-022, and BD1-022S had been integrated into the genomes of the corn plants detected, respectively. Furthermore, single-copy transgenic corn plants had been obtained for the corn plants transformed with the nucleotide sequences of BD1-002, BD1-018, BD1-019, BD1-021, BD1-022, and BD1-022S. The single-copy transgenic corn plants were selected for seed propagation and to produce corn seeds.

The specific method for detecting the copy numbers of the Hpt gene is as follows:
Step 1: respectively collecting 100 mg of leaves from the corn plants transformed with the nucleotide sequence of BD1-002, the corn plants transformed with the nucleotide sequence of BD1-018, the corn plants transformed with the nucleotide sequence of BD1-019, the corn plants transformed with the nucleotide sequence of BD1-022, the corn plants transformed with the nucleotide sequence of BD1-022S, and wild type corn plants, and respectively grinding the leaves into homogenates using liquid nitrogen in a mortar, with three replicates for each sample.
Step 2: extracting genomic DNA from the above samples using the DNeasy Plant Mini Kit from Qiagen (for the specific process, please refer to the product instruction thereof).
Step 3: determining the concentration of the genome DNA of the above samples using the NanoDrop 2000 (Thermo Scientific).
Step 4: adjusting the concentration of the genome DNA to the same concentration ranging from 80 to 100 ng/μL.
Step 5: determining the copy numbers of the samples using the Taqman probe-based fluorescence quantitative PCR assay, with the identified sample with known copy number as a standard and the sample of wild type corn plant as a control; using three replicates for each sample and calculating the mean value.

The sequences of the primers and the probes used in the fluorescence quantitative PCR are as follows:

Primer 5:
(SEQ ID NO: 27)
cagggtgtcacgttgcaaga
Primer 6:
(SEQ ID NO: 28)
ccgctcgtctggctaagatc
Probe 1:
(SEQ ID NO: 29)
tgcctgaaaccgaactgcccgctg

The PCR reaction system is as follows:

| | | |
|---|---|---|
| JumpStart ™ Taq ReadyMix ™ Sigma | 10 | μL |
| 50 × primer/probe mixture | 1 | μL |
| Genomic DNA | 3 | μL |
| ddH₂O | 6 | μL |

The 50× primer/probe mixture contained 45 μl of each primer at a concentration of 1 mM, 50 μl of probe at a concentration of 100 μM, and 860 μl of 1×TE buffer, and was stored in an amber tube at 4° C.

The PCR reaction conditions are as follows:

| Step | Temperature | Time | |
|---|---|---|---|
| 1 | 95 °C | 5 | min |
| 2 | 95 °C | 30 | s |
| 3 | 72 °C | 1 | min |

Repeat steps 1-3 for 40× cycles.

Data were analyzed using IBM SPSS software.

### 4.2. Construction of Transgenic Soybean Plants

### 4.2.1 Construction of Recombinant Vectors

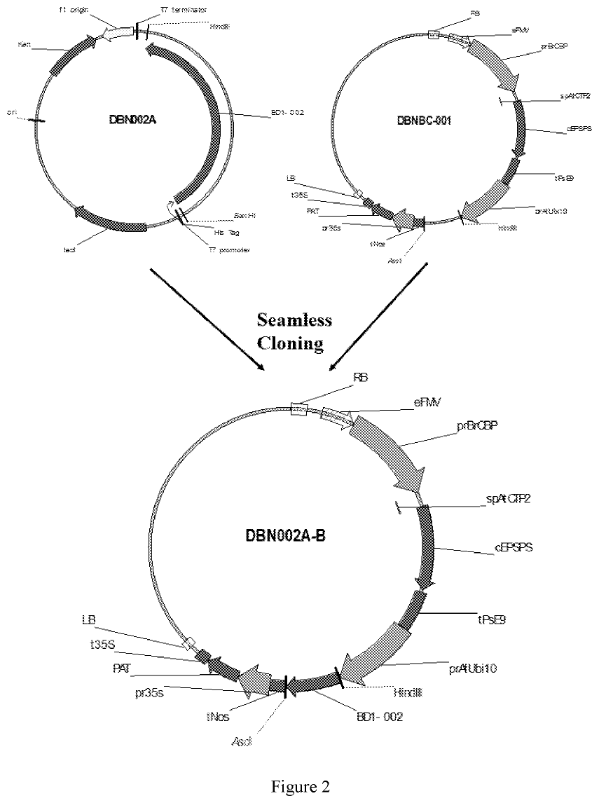

The expression vector DBNBC-001 (vector backbone: pCAMBIA2301, available from the CAMBIA institution) was digested with the restriction enzymes Asc I and Hind III. The nucleotide sequence of BD1-002 was amplified using primer 1 (SEQ ID NO: 23) and primer 2 (SEQ ID NO: 24). The BD1-002 nucleotide sequence fragment obtained by amplification was inserted between the restriction sites of the restriction enzymes Asc I and Hind III in the expression vector DBNBC-001 by seamless cloning to construct the recombinant expression vector DBN002A-B. The construction process is shown in FIG. 2 (RB: right border; eFMV: enhancer; prBrCBP: CBP1 gene promoter; spAtCTP2: signal peptide; cEPSPS: 5-enolpyruvylshikimate-3-phosphate synthase; tPsE9: pea ribulose-1,5-diphosphate carboxylase/oxygenase small subunit E9 protein gene terminator; prAtUbi10: Arabidopsis ubiquitin gene promoter; BD1-002: the nucleotide sequence of BD1-002 (SEQ ID NO: 13); tNos: nopaline synthase (nos) terminator; pr35s: cauliflower mosaic virus 35S promoter; PAT: phosphinothricin acetyltransferase gene; t35s: cauliflower mosaic virus 35S terminator; LB: left border). The method of constructing the vector is well known to those skilled in the art.

The recombinant cloning vector DBN002A-B was transformed into E. coli T1 competent cells through heat shock method. The heat shock conditions were as follows: 50 μL of E. coli T1 competent cell and 10 μL of plasmid DNA were incubated in water bath at 42° C. for 30s; cultured by shaking at 37° C. for 1h (shaking at 100 rpm on a shaking incubator); then the cultured products were plated on a LB solid plate containing 50 mg/L Kanamycin and cultured at 37° C. for 12 h. The single colonies were picked up and inoculated into 5 mL LB liquid medium supplemented with kanamycin at a final concentration of 50 mg/L, and cultured on a shaking incubator (220 rpm) at 37° C. for 16 h. The single colonies were picked up and inoculated into LB liquid medium supplemented with kanamycin at a final concentration of 50 mg/L, and cultured at 37° C. overnight. The plasmid was extracted using the AxyPrep plasmid DNA extraction kit. The extracted plasmids were identified by digestion with the restriction enzymes Asc I and HindIII, and the positive clones were identified through sequencing. The results showed that the nucleotide sequence between the Asc I and HindIII restriction sites in the recombinant expression vector DBN002A-B contained the nucleotide sequence as shown in SEQ ID NO: 13 in the sequence listing, i.e., the nucleotide sequence of BD1-002.

Following the above process for constructing DBN002A-B, the nucleotide sequence of BD1-018 was amplified using primer 1 (SEQ ID NO: 23) and primer 3 (SEQ ID NO: 25). The BD1-018 nucleotide sequence fragment obtained by amplification was inserted into the restriction sites of the expression vector DBNBC-001 by seamless cloning to obtain the recombinant expression vector DBN018A-B. Enzymatic digestion and sequencing verified that the nucleotide sequence in the recombinant expression vector DBN018A-B contained the nucleotide sequence as shown in SEQ ID NO: 18 in the sequence listing, i.e., the nucleotide sequence of BD1-018.

Following the above process for constructing DBN002A-B, the nucleotide sequence of BD1-019 was amplified using primer 1 (SEQ ID NO: 23) and primer 3 (SEQ ID NO: 25). The BD1-019 nucleotide sequence fragment obtained by amplification was inserted into the restriction sites of the expression vector DBNBC-001 by seamless cloning to obtain the recombinant expression vector DBN019A-B. Enzymatic digestion and sequencing verified that the nucleotide sequence in the recombinant expression vector DBN019A-B contained the nucleotide sequence as shown in SEQ ID NO: 19 in the sequence listing, i.e., the nucleotide sequence of BD1-019.

Following the above process for constructing DBN002A-B, the nucleotide sequence of BD1-021 was amplified using primer 1 (SEQ ID NO: 23) and primer 3 (SEQ ID NO: 25). The BD1-021 nucleotide sequence fragment obtained by amplification was inserted into the restriction sites of the expression vector DBNBC-001 by seamless cloning to obtain the recombinant expression vector DBN021A-B. Enzymatic digestion and sequencing verified that the nucleotide sequence in the recombinant expression vector DBN021A-B contained the nucleotide sequence as shown in SEQ ID NO: 21 in the sequence listing, i.e., the nucleotide sequence of BD1-021.

Following the above process for constructing DBN002A-B, the nucleotide sequence of BD1-022 was amplified using primer 1 (SEQ ID NO: 23) and primer 4 (SEQ ID NO: 26). The BD1-022 nucleotide sequence fragment obtained by amplification was inserted into the restriction sites of the expression vector DBNBC-001 by seamless cloning to obtain the recombinant expression vector DBN022A-B. Enzymatic digestion and sequencing verified that the nucleotide sequence in the recombinant expression vector DBN022A-B contained the nucleotide sequence as shown in SEQ ID NO: 22 in the sequence listing, i.e., the nucleotide sequence of BD1-022.

Following the above process for constructing DBN002A-B, the nucleotide sequence of BD1-022S was amplified using primer 1 (SEQ ID NO: 23) and primer 9 (SEQ ID NO: 35). The BD1-022S nucleotide sequence fragment obtained by amplification was inserted into the restriction sites of the expression vector DBNBC-001 by seamless cloning to obtain the recombinant expression vector DBN022SA-B. Enzymatic digestion and sequencing verified that the nucleotide sequence in the recombinant expression vector DBN022SA-B contained the nucleotide sequence as shown in SEQ ID NO: 34 in the sequence listing, i.e., the nucleotide sequence of BD1-022S.

4.2.2 Transformation of Agrobacterium with the Recombinant Expression Vectors

The correctly constructed recombinant expression vectors DBN002A-B, DBN018A-B, DBN019A-B, DBN021A-B, DBN022A-B, and DBN022SA-B were transformed into Agrobacterium tumefaciens strain LBA4404 (InVitrogen, Chicago, USA, CAT: 18313-015) using the liquid nitrogen method. The transformation conditions were as follows: 100 μL Agrobacterium tumefaciens strain LBA4404 and 3 μL plasmid DNA (recombinant expression vector) were placed into liquid nitrogen for 10 min and then incubated in water bath at 37° C. for 10 min. The transformed Agrobacterium tumefaciens strain LBA4404 was inoculated into an LB tube and cultured by shaking (200 rpm) at 28° C. for 2 hours, and then plated on a LB plate containing 50 mg/L of Rifampicin and 100 mg/L of Kanamycin until positive single colonies appeared. The single colonies were picked up and cultured, and the plasmids thereof were extracted. The recombinant expression vectors DBN002A-B, DBN018A-B, DBN019A-B, DBN021A-B, DBN022A-B, and DBN022SA-B were verified by enzymatic digestion with restriction enzymes. The results showed that the structures of the recombinant expression vectors DBN002A-B, DBN018A-B, DBN019A-B, DBN021A-B, DBN022A-B, and DBN022SA-B were correct.

### 4.2.3 Agrobacterium-Mediated Transformation of Soybean Plants

According to the conventional Agrobacterium-mediated transformation method, the sterilely cultured cotyledonary node tissue of the soybean cultivar SY2043-C were co-cultured with the Agrobacterium strains transformed with the recombinant expression vectors so as to transform the T-DNA into the recombinant expression vector DBN002A-B into the soybean genome and then obtain the corn plants into which the nucleotide sequence of BD1-002 was transformed, with wild type soybean plants as control.

Following the above method of obtaining the soybean plants containing the BD1-002, the soybean plants into which the nucleotide sequences of BD1-018, BD1-019, BD1-021, BD1-022, and BD1-022S were transformed were obtained.

As to the Agrobacterium-mediated transformation of soybean plants, in brief, mature soybean seeds were germinated on soybean germination medium (3 g/L of B5 salt, B5 vitamin, 20 g/L of sucrose, and 8 g/L of agar, pH=5.6). The seeds were inoculated onto the germination medium and cultured under the following conditions: 25±1° C., a photoperiod (light/dark) of 16/8h. After 4 to 6 days of germination, the sterile fresh green soybean seedlings with the swollen cotyledonary node were collected. The hypocotyl was cut off at 3 to 4 mm below the cotyledonary node. The cotyledon was longitudinally split, and the apical bud, lateral bud and seminal root were removed. The cotyledonary node was wounded using the back of a scalpel blade and then the wounded cotyledonary node was contacted with Agrobacterium suspension, wherein the Agrobacterium could deliver the nucleotide sequence of Cry1Da1 into the wounded cotyledonary node tissue (Step 1: infection step). In this step, preferably, the cotyledonary node tissue was immersed in the Agrobacterium suspension (OD₆₆₀=0.5~0.8), infection medium (2 g/L of MS salt, B5 vitamins, 20 g/L of sucrose, 10 g/L of glucose, 4 g/L of 2-morpholino ethanesulfonic acid (MES), 2 mg/L of zeatin (ZT), 40 mg/L of acetosyringone, pH 5.3) to initiate the inoculation. The cotyledonary node tissue and Agrobacterium were co-cultured for a period (3 days) (Step 2: co-culture step). Preferably, after the infection step, the cotyledonary node tissue was cultured on solid medium (4 g/L of MS salt, B5 vitamins, 20 g/L of sucrose, 10 g/L of glucose, 8 g/L of agar, 4 g/L of MES, 2 mg/L of ZT, pH=5.6). After this co-culture stage, an optional “recovery” step could be performed. In the “recovery” step, the recovery medium (3 g/L of B5 salt, B5 vitamins, 8 g/L of agar, 30 g/L of sucrose, 1 g/L of MES, 2 mg/L of ZT, 150 mg/L of cephamycin, 100 mg/L of glutamic acid, 100 mg/L of aspartic acid, pH=5.6) contains at least one antibiotic (cephamycin) known to inhibit Agrobacterium growth, but no selective agent for plant transformants (Step 3: recovery step). Preferably, the tissue mass regenerated from the cotyledonary node was cultured on solid medium containing antibiotics but no selective agents so as to eliminate Agrobacterium and to provide a recovery period for the infected cells. Then, the tissue mass regenerated from the cotyledonary node was inoculated and cultured on a medium containing a selective agent (phosphinothricin) and the growing transformed callus was selected (Step 4: selection step). Preferably, the tissue mass regenerated from the cotyledonary node was cultured on selective solid medium containing a selective agent (30 g/L of sucrose, 8 g/L of agar, 3 g/L of B5 salt, B5 vitamins, 1 g/L of MES, 1 mg/L of 6-benzyladenine, 150 mg/L of cephamycin, 100 mg/L of glutamic acid, 100 mg/L of aspartic acid, 6 mg/L of phosphinothricin, pH=5.6), resulting in selective growth of the transformed cells. Then, the transformed cells regenerated into plants (Step 5: regeneration step). Preferably, the tissue mass regenerated from the cotyledonary node growing on medium containing s selective agent was cultured on solid medium (B5 differentiation medium and B5 rooting medium) to regenerate plants.

The resistant tissue mass obtained by selection was transferred to the B5 differentiation medium (3.1 g/L B5 salt, B5 vitamins, 1 g/L of MES, 30 g/L of sucrose, 1 mg/L of ZT, 8 g/L of agar, 150 mg/L of cephamycin, 50 mg/L of glutamic acid, 50 mg/L of aspartic acid, 1 mg/L of gibberellin, 1 mg/L of auxin, 6 mg/L of phosphinothricin, pH-5.6) and cultured at 25° C. for differentiation. The differentiated seedlings were transferred to the B5 rooting medium (3.1 g/L of B5 salt, B5 vitamins, 1 g/L of MES, 30 g/L of sucrose, 8 g/L of agar, 150 mg/L of cephamycin, 1 mg/L of indole-3-butyric acid) and cultured at 25° C. for rooting culture until they reached about 10 cm in height, and then, they were moved to a greenhouse and cultured until fructification. In the greenhouse, the plants were cultured at 26° C. for 16 h and at 20° C. for 8 h every day.

### 4.2.4 Identification of Transgenic Soybean Materials

About 100 mg of leaves from soybean plant transformed with the nucleotide sequences of BD1-002, BD1-018, BD1-019, BD1-021, BD1-022, and BD1-022S were respectively taken as sample. Genomic DNA was extracted using the DNeasy Plant Maxi Kit from Qiagen and the copy numbers of the PAT gene were detected using the Taqman probe-based fluorescence quantitative PCR assay to determine the copy numbers of the Cry1Da1 gene. Wild type SY2043C soybean plants were taken as controls. The analysis was performed according to the above process. The experiment was carried out three times. The analysis of the experimental results of the copy number of the target gene showed that the nucleotide sequences of BD1-002, BD1-018, BD1-019, BD1-021, BD1-022, and BD1-022S had been integrated into the genomes of the soybean plants detected, respectively. Furthermore, single-copy transgenic soybean plants had been obtained for the soybean plants transformed with the nucleotide sequences of BD1-002, BD1-018, BD1-019, BD1-021, BD1-022, and BD1-022S. The single-copy transgenic soybean plants were selected for seed propagation to produce soybean seeds.

The specific method for detecting the copy numbers of the PAT gene is as follows:
Step 1: respectively collecting 100 mg of leaves from the soybean plant transformed with the nucleotide sequence of BD1-002, the soybean plant transformed with the nucleotide sequence of BD1-018, the soybean plant transformed with the nucleotide sequence of BD1-019, the soybean plant transformed with the nucleotide sequence of BD1-022, the soybean plant transformed with the nucleotide sequence of BD1-022S, and wild type soybean plant, and respectively grinding the leaves into homogenates using liquid nitrogen in a mortar, with three replicates for each sample.
Step 2: extracting genomic DNA from the above samples using the DNeasy Plant Mini Kit from Qiagen (for the specific process, please refer to the product instruction thereof).
Step 3: determining the concentration of the genome DNA of the above samples using the NanoDrop 2000 (Thermo Scientific).
Step 4: adjusting the concentration of the genome DNA to the same concentration ranging from 80 to 100 ng/μL.
Step 5: determining the copy numbers of the samples using the Taqman probe-based fluorescence quantitative PCR assay, with the identified sample with known copy number as a standard and the sample of wild type soybean plant as a control; using three replicates for each sample and calculating the mean value.

The sequences of the primers and the probes used in the fluorescence quantitative PCR are as follows:

Primer 7:
(SEQ ID NO: 30)
gagggtgttgtggctggtattg
Primer 8:
(SEQ ID NO: 31)
tctcaactgtccaatcgtaagcg
Probe 2:
(SEQ ID NO: 32)
cttacgctgggccctggaaggctag

The PCR reaction system is as follows:

| | | |
|---|---|---|
| JumpStart ™ Taq ReadyMix ™ Sigma | 10 | μL |
| 50 × primer/probe mixture | 1 | μL |
| Genomic DNA | 3 | μL |
| ddH₂O | 6 | μL |

The 50× primer/probe mixture contained 45 μl of each primer at a concentration of 1 mM, 50 μl of probe at a concentration of 100 μM, and 860 μl of 1×TE buffer, and was stored in an amber tube at 4° C.

The PCR reaction conditions are as follows:

| Step | Temperature | Time | |
|---|---|---|---|
| 1 | 95 °C | 5 | min |
| 2 | 95 °C | 30 | s |
| 3 | 72 °C | 1 | min |

Repeat steps 1-3 for 40× cycles.

Data were analyzed using IBM SPSS software.

### Example 5: Activity Test of Transgenic Corn Against Lepidoptera Pests

5.1 Insect-Resistant Effect Against Spodoptera frugiperda

When the transgenic corn plants expressing the modified protein grew and reached the V3-V4 stage, fresh corn leaves were collected and used for the Spodoptera frugiperda bioassay. The leaves were rinsed with sterile water and the water on the leaves was absorbed to dryness by gauze. The leaves were cut into strips of about 2 cm×3.5 cm. One strip of leaves was placed on moisturizing filter paper at the bottom of a round plastic Petri dish. Ten newly hatched larvae of Spodoptera frugiperda were put into each Petri dish. The Petri dishes for insect test were covered and placed under the following conditions for one day: 25-28° C., relative humidity 70% and a photoperiod (light/dark) of 16:8 hours. The mortality of Spodoptera frugiperda larvae and leaf damage rate were calculated and collected: mortality=(the number of dead larvae/total number of introduced larvae)×100%, with the corn plant having the same genetic background and not being transformed with the insect-resistant protein as control. The corrected mortality was calculated based on the control mortality: corrected mortality=(the treatment mortality−the control mortality)/(1−the control mortality)×100%. The results are shown in Table 6.

TABLE 6
The results of the insect-resistant experiments of the transgenic
corn plants inoculated with Spodoptera frugiperda (1 day)

| Protein number | Corrected mortality % |
|---|---|
| BD1-002 | 86.6 ± 6.58 |
| BD1-018 | 90.7 ± 4.04 |
| BD1-019 | 89.3 ± 6.27 |
| BD1-021 | 87.3 ± 4.73 |
| BD1-022 | 90.7 ± 5.34 |
| BD1-022S | 91.3 ± 6.51 |

5.2 Insect-Resistant Effect of Transgenic Corn Plants Against Helicoverpa armigera

When the transgenic corn plants expressing the modified protein grew and reached the V3-V4 stage, fresh corn leaves were collected and used for the Helicoverpa armigera bioassay. The leaves were rinsed with sterile water and the water on the leaves was absorbed to dryness by gauze. The leaves were cut into strips of about 2 cm×3.5 cm. One strip of leaves was placed on moisturizing filter paper at the bottom of a round plastic Petri dish. Ten newly hatched larvae of Helicoverpa armigera were put into each Petri dish. The Petri dishes for insect test were covered and placed under the following conditions for 3 days: 25-28° C., relative humidity 70% and a photoperiod (light/dark) of 16:8 hours. The mortality of Helicoverpa armigera larvae was calculated and collected: mortality=(the number of dead larvae/total number of introduced larvae)×100%, with the corn plant having the same genetic background and not being transformed with the insect-resistant protein as control. The corrected mortality was calculated based on the control mortality: corrected mortality=(the treatment mortality−the control mortality)/(1−the control mortality)×100%. The results are shown in Table 7.

TABLE 7
The results of the insect-resistant experiments of the transgenic
corn plants inoculated with Helicoverpa armigera (3 days)

| Protein number | Corrected mortality % |
|---|---|
| BD1-002 | 59.2 ± 10.68 |
| BD1-018 | 67.3 ± 8.02 |
| BD1-019 | 67.5 ± 11.37 |
| BD1-021 | 64.7 ± 10.26 |
| BD1-022 | 63.8 ± 13.65 |
| BD1-022S | 64.7 ± 10.50 |

From Tables 6-7, it can be seen that as compared with the transgenic corn plants expressing the BD1-002 protein, after inoculating Spodoptera frugiperda and Helicoverpa armigera on the corn plants expressing the modified protein of the present application, the mortality of larvae was higher, indicating that the corn expressing the modified protein of the present application had superior insect-resistant effect against Spodoptera frugiperda and Helicoverpa armigera.

### Example 6: Insect-Resistant Effect of Transgenic Soybean Against Lepidoptera Pests

6.1 Insect-Resistant Effect of Transgenic Soybean Plants Against Argyrogramma agnata

When the transgenic soybean plants expressing the modified protein grew and reached the V3 stage, the penultimate leaves were collected and used for the Argyrogramma agnata bioassay. The leaves were rinsed with sterile water and the water on the leaves was absorbed to dryness by gauze. The leaves were cut into strips of about 2 cm×3.5 cm. One strip of leaves was placed on moisturizing filter paper at the bottom of a round plastic Petri dish. Ten newly hatched larvae of Argyrogramma agnata were put into each Petri dish. The Petri dishes for insect test were covered and placed under the following conditions for 3 days: 25-28° C., relative humidity 70% and a photoperiod (light/dark) of 16:8 hours. The mortality of Argyrogramma agnata larvae and leaf damage rate were calculated and collected: mortality=(the number of dead larvae/total number of introduced larvae)×100%. The larvae inhibition rate was calculated: larvae inhibition rate=(quantity of control larvae-quantity of treated larvae of the same larval instar)/quantity of control insect. The soybean plant having the same genetic background and not being transformed with the insect-resistant protein was used as control. The corrected mortality was calculated based on the control mortality: corrected mortality=(the treatment mortality−the control mortality)/(1−the control mortality)×100%. The results are shown in Table 8.

TABLE 8
The bioassay results of soybean leaves
against Argyrogramma agnate (3 days)

| Protein | Average leaf damage | Corrected | Larvae inhibition |
|---|---|---|---|
| number | rate % | mortality % | rate % |
| Control | 56 | // | // |
| BD1-002 | 23 | 23 | 48 |
| BD1-018 | 17 | 28 | 52 |
| BD1-019 | 18 | 27 | 50 |
| BD1-021 | 18 | 26 | 51 |
| BD1-022 | 17 | 29 | 53 |
| BD1-022S | 16 | 29 | 58 |

6.2 Insect-Resistant Effect of Transgenic Soybean Plants Against Spodoptera frugiperda

When the transgenic soybean plants expressing the modified protein grew and reached the V3 stage, the penultimate leaves were collected and used for the Spodoptera frugiperda bioassay. The leaves were rinsed with sterile water and the water on the leaves was absorbed to dryness by gauze. The leaves were cut into strips of about 2 cm×3.5 cm. One strip of leaves was placed on moisturizing filter paper at the bottom of a round plastic Petri dish. Ten newly hatched larvae of Spodoptera frugiperda were put into each Petri dish. The Petri dishes for insect test were covered and placed under the following conditions for 3 days: 25-28° C., relative humidity 70% and a photoperiod (light/dark) of 16:8 hours. The mortality of Spodoptera frugiperda larvae was calculated and collected: mortality=(the number of dead larvae/total number of introduced larvae)×100%, with the corn plant having the same genetic background and not being transformed with the insect-resistant protein as control. The corrected mortality was calculated based on the control mortality: corrected mortality=(the treatment mortality−the control mortality)/(1−the control mortality)×100%. The results are shown in Table 9.

TABLE 9
The bioassay results of soybean leaves against
Spodoptera frugiperda (3 days)

| Protein number | Corrected mortality % |
|---|---|
| BD1-002 | 45% ± 24% |
| BD1-018 | 64% ± 11% |
| BD1-019 | 62% ± 16% |
| BD1-022 | 66% ± 31% |
| BD1-022S | 68% ± 28% |

From Table 8, it can be seen that as compared with the transgenic soybean expressing the BD1-002 protein, the leaf damage rate of the soybean plants expressing the modified protein of the present application was lower, and the mortality and inhibition rate of Argyrogramma agnata larvae were higher, indicating that the soybean expressing the modified protein of the present application had superior insect-resistant effect against Argyrogramma agnata. Similarly, from Table 9, it can be seen that the mortality of the soybean plants expressing the modified protein of the present application against Spodoptera frugiperda was higher than that of the transgenic soybean expressing the BD1-002 protein, indicating that the modified protein of the present application had superior insect-resistant effect against Argyrogramma agnata.

Finally, it should be noted that the above Examples are merely intended to illustrate the technical solutions of present invention rather than to limit the present invention. Although the present invention has been described in detail with reference to preferred Examples, those skilled in the art should understand that modifications or equivalent replacements to the technical solutions of the present invention can be made without departing from the spirit and scope of the technical solutions of the present invention.

### REFERENCE

1. Bacillus thuringiensis Cry1Da_7 and Cry1B.868 Protein Interactions with Novel Receptors Allow Control of Resistant Fall Armyworms, Spodoptera frugiperda (J. E. Smith). (2019) Appl Environ Microbiol 85.

1. An insecticidal protein comprising the amino acid sequence as shown in SEQ ID NO: 7, SEQ ID NO: 8, SEQ ID NO: 10, SEQ ID NO: 11, or SEQ ID NO: 33.

2. A nucleic acid molecule encoding the insecticidal protein according to claim 1;
preferably, the nucleotide sequences encoding the amino acid sequences as shown in SEQ ID NO: 7, SEQ ID NO: 8, SEQ ID NO: 10, SEQ ID NO: 11 or SEQ ID NO: 33, are shown in SEQ ID NO: 18, SEQ ID NO: 19, SEQ ID NO: 21, SEQ ID NO: 22, or SEQ ID NO: 34, respectively.

3. A recombinant expression vector comprising the nucleic acid molecule according to claim 2.

4. An insecticidal composition comprising the insecticidal protein according to claim 1.

5. A method for controlling Lepidoptera pests, comprising contacting the Lepidoptera pests with the insecticidal protein according to claim 1.

6. A method for controlling Lepidoptera pests, comprising introducing the nucleic acid molecule according to claim 2 into a plant, and making Lepidoptera pests feed on the plant.

7. The method according to claim 5, wherein the Lepidoptera pests are Spodoptera frugiperda, Helicoverpa armigera, or Argyrogramma agnata.

8. The method according to claim 6, wherein the plant is a monocotyledon or a dicotyledon; preferably, the plant is corn or soybean.

9.-10. (canceled)

11. A method for controlling Lepidoptera pests, comprising contacting the Lepidoptera pests with the insecticidal composition according to claim 4.

12. A method for controlling Lepidoptera pests, comprising introducing the recombinant expression vector according to claim 3 into a plant, and making Lepidoptera pests feed on the plant.

13. The method according to claim 6, wherein the Lepidoptera pests are Spodoptera frugiperda, Helicoverpa armigera, or Argyrogramma agnata.

14. The method according to claim 11, wherein the Lepidoptera pests are Spodoptera frugiperda, Helicoverpa armigera, or Argyrogramma agnata.

15. The method according to claim 12, wherein the Lepidoptera pests are Spodoptera frugiperda, Helicoverpa armigera, or Argyrogramma agnata.

16. The method according to claim 12, wherein the plant is a monocotyledon or a dicotyledon; preferably, the plant is corn or soybean.


SEQUENCE LISTING (35 sequences)

SEQ ID NO: 1 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAASYPTFSAAESAIIRSPHLVDFLNSFTIYTDSLARYAYWGGHLVNSFRTGTTTNLIRSPLYGREGNTERPVTITASPSVPIFRTLSYITGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 2 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYPTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 3 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAALYPTFSAAESAIIRSPHLVDFLNSFTIYTDSLARSAYWGGHLVNSFRTGTTTNLIRSPLYGREGNTERPVTITASPSVPIFRTLSYPTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 4 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYFTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 5 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAALYPTFSAAESAIIRSPHLVDFLNSFTIYTDSLARSAYWGGHLVNSFRTGTTTNLIRSPLYGREGNTERPVTITASPSVPIFRTLSYFTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 6 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARTAYWGGHLVNSFRTGTTTNLIRSPLYGREGNTERPVTITASPSVPIFRTLSYPTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 7 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYFTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKAVNALFTSTNQLGLKTNVTDYHIDQVSNLVTYLSDEFCLDEKRELSEKVKHAKRLSDERNLLQDPNFRGINRQPDRGWRGSTDITIQGGDDVFKENYVTLSGTFDECYPTYLYQKIDESKLKAYTRYQLRGYIEDSQDLEIYLIRYNAKHETVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAYGNNPSVPADYASVYEEKSYTDGRRENPCEFNRGYRDYTPLPVGYVTKELEYFPETDKVWIEIGETEGTFIVDSVELLLMEE

SEQ ID NO: 8 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYFTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATLEAEYNLERAQKAVNALFTSTNQLGLKTNVTDYHIDQVSNLVTYLSDEFCLDEKRELSEKVKHAKRLSDERNLLQDPNFRGINRQPDRGWRGSTDITIQGGDDVFKENYVTLSGTFDECYPTYLYQKIDESKLKAYTRYQLRGYIEDSQDLEIYLIRYNAKHETVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAYGNNPSVPADYASVYEEKSYTDGRRENPCEFNRGYRDYTPLPVGYVTKELEYFPETDKVWIEIGETEGTFIVDSVELLLMEE

SEQ ID NO: 9 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYYTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKAVNALFTSTNQLGLKTNVTDYHIDQVSNLVTYLSDEFCLDEKRELSEKVKHAKRLSDERNLLQDPNFRGINRQPDRGWRGSTDITIQGGDDVFKENYVTLSGTFDECYPTYLYQKIDESKLKAYTRYQLRGYIEDSQDLEIYLIRYNAKHETVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAYGNNPSVPADYASVYEEKSYTDGRRENPCEFNRGYRDYTPLPVGYVTKELEYFPETDKVWIEIGETEGTFIVDSVELLLMEE

SEQ ID NO: 10 - protein, 1165 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYWTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKAVNALFTSTNQLGLKTNVTDYHIDQVSNLVTYLSDEFCLDEKRELSEKVKHAKRLSDERNLLQDPNFRGINRQPDRGWRGSTDITIQGGDDVFKENYVTLSGTFDECYPTYLYQKIDESKLKAYTRYQLRGYIEDSQDLEIYLIRYNAKHETVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAYGNNPSVPADYASVYEEKSYTDGRRENPCEFNRGYRDYTPLPVGYVTKELEYFPETDKVWIEIGETEGTFIVDSVELLLMEE

SEQ ID NO: 11 - protein, 1161 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYFTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 12 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcatcctatccaaccttttctgccgctgaaagcgcaataattcgtagtccacatctggttgactttttaaattccttcacaatctataccgatagcctagcacggtatgcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctatattacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagttgtgaatgcgttgtttactagcaccaaccaacttggactcaaaacagatgttaccgactaccatatagatcaggtttccaacctcgttgcgtgtctcagcgatgagttctgtttggatgaaaaaagagaactgtcagagaaagttaaacatgcgaaacgtctcagcgacgaacgaaatctgctgcaagatcccaattttcgtggtattaatcgtcagcctgatcgtgggtggcgtggttccacggatatcaccattcagggtggtgatgatgttttcaaggaaaattatgttacactgccaggaacctttgatgagtgctacccgacatatctgtaccagaagattgatgaatcaaaactgaaagcatatacaagatatcagctccgtgggtacatagaggactctcaggatctagagatttatctaatccgctataatgcgaagcatgaaatagttaatgttcctggtactggatctctgtggccgctgagcgttgagaatcagattggtccttgtggagagcctaatagatgtgcaccgcacctcgagtggaaccccgacctccattgctcgtgtcgtgatggagagaaatgtgcacatcacagccaccatttcagcctggatattgacgttggctgcacagatctgaatgaagacctcggcgtgtgggttatctttaaaattaaaactcaggatggtcatgcaagactcggcaatctcgaatttctggaggaaaaaccgctgctgggtgaagcattagctcgtgtgaagcgtgcagaaaagaagtggagagataaacgtgagacactgcagctggaaactaccattgtgtacaaagaagcaaaagaaagcgttgatgcgctctttgtaaactcgcagtacgatcgtctgcaggcagatacaaacattgccatgattcatgctgcggataaacgtgtacaccgaattcgtgaagcatatctcccagaactgagcgtgatcccgggcgtgaatgcagcaatctttgaagaattggaagaacgtatatttactgccttctctttgtatgacgcacggaacattattaaaaatggagatttcaacaatggcctcctgtgctggaatgtgaaaggccatgtggaagttgaggagcagaataaccatcggtccgttctggttattcctgaatgggaagccgaggtttcacaagaagttcgcgtctgtccgggtcgtggttacattctgcgcgttaccgcgtacaaagaaggttatggtgaaggttgcgttacaatccatgagatcgagaacaatacagatgaactgaaattcaataattgcgtcgaagaggaggtatacccaaacaatactgttacgtgcataaattacaccgcaacgcaggaggagtacgaaggtacgtataccagccgcaatcgtggctatgacgaagcatatggaaacaacccgagcgttccggcggattatgctagcgtctatgaagaaaaatcttacaccgatcgtcgtcgcgagaacccgtgtgaaagcaacaggggttacggagattataccccgctgcccgcgggctatgtaaccaaagagctggaatattttccggagaccgacaaagtgtggattgaaattggcgaaactgaaggcacctttatcgtagattctgtagaactgctgctgatggaggaataa

SEQ ID NO: 13 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctatccgacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagttgtgaatgcgttgtttactagcaccaaccaacttggactcaaaacagatgttaccgactaccatatagatcaggtttccaacctcgttgcgtgtctcagcgatgagttctgtttggatgaaaaaagagaactgtcagagaaagttaaacatgcgaaacgtctcagcgacgaacgaaatctgctgcaagatcccaattttcgtggtattaatcgtcagcctgatcgtgggtggcgtggttccacggatatcaccattcagggtggtgatgatgttttcaaggaaaattatgttacactgccaggaacctttgatgagtgctacccgacatatctgtaccagaagattgatgaatcaaaactgaaagcatatacaagatatcagctccgtgggtacatagaggactctcaggatctagagatttatctaatccgctataatgcgaagcatgaaatagttaatgttcctggtactggatctctgtggccgctgagcgttgagaatcagattggtccttgtggagagcctaatagatgtgcaccgcacctcgagtggaaccccgacctccattgctcgtgtcgtgatggagagaaatgtgcacatcacagccaccatttcagcctggatattgacgttggctgcacagatctgaatgaagacctcggcgtgtgggttatctttaaaattaaaactcaggatggtcatgcaagactcggcaatctcgaatttctggaggaaaaaccgctgctgggtgaagcattagctcgtgtgaagcgtgcagaaaagaagtggagagataaacgtgagacactgcagctggaaactaccattgtgtacaaagaagcaaaagaaagcgttgatgcgctctttgtaaactcgcagtacgatcgtctgcaggcagatacaaacattgccatgattcatgctgcggataaacgtgtacaccgaattcgtgaagcatatctcccagaactgagcgtgatcccgggcgtgaatgcagcaatctttgaagaattggaagaacgtatatttactgccttctctttgtatgacgcacggaacattattaaaaatggagatttcaacaatggcctcctgtgctggaatgtgaaaggccatgtggaagttgaggagcagaataaccatcggtccgttctggttattcctgaatgggaagccgaggtttcacaagaagttcgcgtctgtccgggtcgtggttacattctgcgcgttaccgcgtacaaagaaggttatggtgaaggttgcgttacaatccatgagatcgagaacaatacagatgaactgaaattcaataattgcgtcgaagaggaggtatacccaaacaatactgttacgtgcataaattacaccgcaacgcaggaggagtacgaaggtacgtataccagccgcaatcgtggctatgacgaagcatatggaaacaacccgagcgttccggcggattatgctagcgtctatgaagaaaaatcttacaccgatcgtcgtcgcgagaacccgtgtgaaagcaacaggggttacggagattataccccgctgcccgcgggctatgtaaccaaagagctggaatattttccggagaccgacaaagtgtggattgaaattggcgaaactgaaggcacctttatcgtagattctgtagaactgctgctgatggaggaataa

SEQ ID NO: 14 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcactgtatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctatccgacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagttgtgaatgcgttgtttactagcaccaaccaacttggactcaaaacagatgttaccgactaccatatagatcaggtttccaacctcgttgcgtgtctcagcgatgagttctgtttggatgaaaaaagagaactgtcagagaaagttaaacatgcgaaacgtctcagcgacgaacgaaatctgctgcaagatcccaattttcgtggtattaatcgtcagcctgatcgtgggtggcgtggttccacggatatcaccattcagggtggtgatgatgttttcaaggaaaattatgttacactgccaggaacctttgatgagtgctacccgacatatctgtaccagaagattgatgaatcaaaactgaaagcatatacaagatatcagctccgtgggtacatagaggactctcaggatctagagatttatctaatccgctataatgcgaagcatgaaatagttaatgttcctggtactggatctctgtggccgctgagcgttgagaatcagattggtccttgtggagagcctaatagatgtgcaccgcacctcgagtggaaccccgacctccattgctcgtgtcgtgatggagagaaatgtgcacatcacagccaccatttcagcctggatattgacgttggctgcacagatctgaatgaagacctcggcgtgtgggttatctttaaaattaaaactcaggatggtcatgcaagactcggcaatctcgaatttctggaggaaaaaccgctgctgggtgaagcattagctcgtgtgaagcgtgcagaaaagaagtggagagataaacgtgagacactgcagctggaaactaccattgtgtacaaagaagcaaaagaaagcgttgatgcgctctttgtaaactcgcagtacgatcgtctgcaggcagatacaaacattgccatgattcatgctgcggataaacgtgtacaccgaattcgtgaagcatatctcccagaactgagcgtgatcccgggcgtgaatgcagcaatctttgaagaattggaagaacgtatatttactgccttctctttgtatgacgcacggaacattattaaaaatggagatttcaacaatggcctcctgtgctggaatgtgaaaggccatgtggaagttgaggagcagaataaccatcggtccgttctggttattcctgaatgggaagccgaggtttcacaagaagttcgcgtctgtccgggtcgtggttacattctgcgcgttaccgcgtacaaagaaggttatggtgaaggttgcgttacaatccatgagatcgagaacaatacagatgaactgaaattcaataattgcgtcgaagaggaggtatacccaaacaatactgttacgtgcataaattacaccgcaacgcaggaggagtacgaaggtacgtataccagccgcaatcgtggctatgacgaagcatatggaaacaacccgagcgttccggcggattatgctagcgtctatgaagaaaaatcttacaccgatcgtcgtcgcgagaacccgtgtgaaagcaacaggggttacggagattataccccgctgcccgcgggctatgtaaccaaagagctggaatattttccggagaccgacaaagtgtggattgaaattggcgaaactgaaggcacctttatcgtagattctgtagaactgctgctgatggaggaataa

SEQ ID NO: 15 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattttacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagttgtgaatgcgttgtttactagcaccaaccaacttggactcaaaacagatgttaccgactaccatatagatcaggtttccaacctcgttgcgtgtctcagcgatgagttctgtttggatgaaaaaagagaactgtcagagaaagttaaacatgcgaaacgtctcagcgacgaacgaaatctgctgcaagatcccaattttcgtggtattaatcgtcagcctgatcgtgggtggcgtggttccacggatatcaccattcagggtggtgatgatgttttcaaggaaaattatgttacactgccaggaacctttgatgagtgctacccgacatatctgtaccagaagattgatgaatcaaaactgaaagcatatacaagatatcagctccgtgggtacatagaggactctcaggatctagagatttatctaatccgctataatgcgaagcatgaaatagttaatgttcctggtactggatctctgtggccgctgagcgttgagaatcagattggtccttgtggagagcctaatagatgtgcaccgcacctcgagtggaaccccgacctccattgctcgtgtcgtgatggagagaaatgtgcacatcacagccaccatttcagcctggatattgacgttggctgcacagatctgaatgaagacctcggcgtgtgggttatctttaaaattaaaactcaggatggtcatgcaagactcggcaatctcgaatttctggaggaaaaaccgctgctgggtgaagcattagctcgtgtgaagcgtgcagaaaagaagtggagagataaacgtgagacactgcagctggaaactaccattgtgtacaaagaagcaaaagaaagcgttgatgcgctctttgtaaactcgcagtacgatcgtctgcaggcagatacaaacattgccatgattcatgctgcggataaacgtgtacaccgaattcgtgaagcatatctcccagaactgagcgtgatcccgggcgtgaatgcagcaatctttgaagaattggaagaacgtatatttactgccttctctttgtatgacgcacggaacattattaaaaatggagatttcaacaatggcctcctgtgctggaatgtgaaaggccatgtggaagttgaggagcagaataaccatcggtccgttctggttattcctgaatgggaagccgaggtttcacaagaagttcgcgtctgtccgggtcgtggttacattctgcgcgttaccgcgtacaaagaaggttatggtgaaggttgcgttacaatccatgagatcgagaacaatacagatgaactgaaattcaataattgcgtcgaagaggaggtatacccaaacaatactgttacgtgcataaattacaccgcaacgcaggaggagtacgaaggtacgtataccagccgcaatcgtggctatgacgaagcatatggaaacaacccgagcgttccggcggattatgctagcgtctatgaagaaaaatcttacaccgatcgtcgtcgcgagaacccgtgtgaaagcaacaggggttacggagattataccccgctgcccgcgggctatgtaaccaaagagctggaatattttccggagaccgacaaagtgtggattgaaattggcgaaactgaaggcacctttatcgtagattctgtagaactgctgctgatggaggaataa

SEQ ID NO: 16 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcactgtatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattttacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagttgtgaatgcgttgtttactagcaccaaccaacttggactcaaaacagatgttaccgactaccatatagatcaggtttccaacctcgttgcgtgtctcagcgatgagttctgtttggatgaaaaaagagaactgtcagagaaagttaaacatgcgaaacgtctcagcgacgaacgaaatctgctgcaagatcccaattttcgtggtattaatcgtcagcctgatcgtgggtggcgtggttccacggatatcaccattcagggtggtgatgatgttttcaaggaaaattatgttacactgccaggaacctttgatgagtgctacccgacatatctgtaccagaagattgatgaatcaaaactgaaagcatatacaagatatcagctccgtgggtacatagaggactctcaggatctagagatttatctaatccgctataatgcgaagcatgaaatagttaatgttcctggtactggatctctgtggccgctgagcgttgagaatcagattggtccttgtggagagcctaatagatgtgcaccgcacctcgagtggaaccccgacctccattgctcgtgtcgtgatggagagaaatgtgcacatcacagccaccatttcagcctggatattgacgttggctgcacagatctgaatgaagacctcggcgtgtgggttatctttaaaattaaaactcaggatggtcatgcaagactcggcaatctcgaatttctggaggaaaaaccgctgctgggtgaagcattagctcgtgtgaagcgtgcagaaaagaagtggagagataaacgtgagacactgcagctggaaactaccattgtgtacaaagaagcaaaagaaagcgttgatgcgctctttgtaaactcgcagtacgatcgtctgcaggcagatacaaacattgccatgattcatgctgcggataaacgtgtacaccgaattcgtgaagcatatctcccagaactgagcgtgatcccgggcgtgaatgcagcaatctttgaagaattggaagaacgtatatttactgccttctctttgtatgacgcacggaacattattaaaaatggagatttcaacaatggcctcctgtgctggaatgtgaaaggccatgtggaagttgaggagcagaataaccatcggtccgttctggttattcctgaatgggaagccgaggtttcacaagaagttcgcgtctgtccgggtcgtggttacattctgcgcgttaccgcgtacaaagaaggttatggtgaaggttgcgttacaatccatgagatcgagaacaatacagatgaactgaaattcaataattgcgtcgaagaggaggtatacccaaacaatactgttacgtgcataaattacaccgcaacgcaggaggagtacgaaggtacgtataccagccgcaatcgtggctatgacgaagcatatggaaacaacccgagcgttccggcggattatgctagcgtctatgaagaaaaatcttacaccgatcgtcgtcgcgagaacccgtgtgaaagcaacaggggttacggagattataccccgctgcccgcgggctatgtaaccaaagagctggaatattttccggagaccgacaaagtgtggattgaaattggcgaaactgaaggcacctttatcgtagattctgtagaactgctgctgatggaggaataa

SEQ ID NO: 17 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggaccgcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctatccgacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagttgtgaatgcgttgtttactagcaccaaccaacttggactcaaaacagatgttaccgactaccatatagatcaggtttccaacctcgttgcgtgtctcagcgatgagttctgtttggatgaaaaaagagaactgtcagagaaagttaaacatgcgaaacgtctcagcgacgaacgaaatctgctgcaagatcccaattttcgtggtattaatcgtcagcctgatcgtgggtggcgtggttccacggatatcaccattcagggtggtgatgatgttttcaaggaaaattatgttacactgccaggaacctttgatgagtgctacccgacatatctgtaccagaagattgatgaatcaaaactgaaagcatatacaagatatcagctccgtgggtacatagaggactctcaggatctagagatttatctaatccgctataatgcgaagcatgaaatagttaatgttcctggtactggatctctgtggccgctgagcgttgagaatcagattggtccttgtggagagcctaatagatgtgcaccgcacctcgagtggaaccccgacctccattgctcgtgtcgtgatggagagaaatgtgcacatcacagccaccatttcagcctggatattgacgttggctgcacagatctgaatgaagacctcggcgtgtgggttatctttaaaattaaaactcaggatggtcatgcaagactcggcaatctcgaatttctggaggaaaaaccgctgctgggtgaagcattagctcgtgtgaagcgtgcagaaaagaagtggagagataaacgtgagacactgcagctggaaactaccattgtgtacaaagaagcaaaagaaagcgttgatgcgctctttgtaaactcgcagtacgatcgtctgcaggcagatacaaacattgccatgattcatgctgcggataaacgtgtacaccgaattcgtgaagcatatctcccagaactgagcgtgatcccgggcgtgaatgcagcaatctttgaagaattggaagaacgtatatttactgccttctctttgtatgacgcacggaacattattaaaaatggagatttcaacaatggcctcctgtgctggaatgtgaaaggccatgtggaagttgaggagcagaataaccatcggtccgttctggttattcctgaatgggaagccgaggtttcacaagaagttcgcgtctgtccgggtcgtggttacattctgcgcgttaccgcgtacaaagaaggttatggtgaaggttgcgttacaatccatgagatcgagaacaatacagatgaactgaaattcaataattgcgtcgaagaggaggtatacccaaacaatactgttacgtgcataaattacaccgcaacgcaggaggagtacgaaggtacgtataccagccgcaatcgtggctatgacgaagcatatggaaacaacccgagcgttccggcggattatgctagcgtctatgaagaaaaatcttacaccgatcgtcgtcgcgagaacccgtgtgaaagcaacaggggttacggagattataccccgctgcccgcgggctatgtaaccaaagagctggaatattttccggagaccgacaaagtgtggattgaaattggcgaaactgaaggcacctttatcgtagattctgtagaactgctgctgatggaggaataa

SEQ ID NO: 18 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattttacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagccgtgaacgcattgtttacaagcaccaaccaactgggtctgaaaacgaatgtaaccgactaccacatagatcaggtcagtaatctggtaacatatctgtccgatgagttctgcctcgatgaaaagagggaactgtctgaaaaggtcaaacatgccaaaagactgagcgatgagaggaacctccttcaggacccgaactttcggggaataaatcgccagccggaccgtggatggcgtggtagcaccgacattaccatccagggcggcgacgatgtgttcaaggaaaactacgtcaccctgagcggtacgtttgatgagtgctacccgacttacctttatcagaagatagatgaaagcaagctcaaggcatacactcgctatcagctccgcgggtacattgaggattcacaagatcttgagatttatctcatcagatacaacgcaaaacatgaaaccgttaatgtgccgggcaccggtagcctctggccgctgagcgttgaaaatcaaattggcccgtgtggcgagccgaaccggtgtgcgccacatttggagtggaatccggatctgcactgctcttgtagggacggggaaaaatgtgcccatcattcacatcactttagcctggatattgatgttgggtgtaccgatttaaatgaagatctaggtgtctgggtgatcttcaaaattaaaactcaggacggccacgcacggctgggtaacttggaatttctagaagagaaacccctggtcggagaggcgctggcccgtgttaaacgggcagaaaagaagtggcgtgacaaacgtgagaaactggaatgggagacgaacattgtgtacaaggaggcaaaagaaagcgttgatgcgctctttgtgaattctcagtacgaccagctgcaggcagatactaatattgccatgatacatgctgcagacaagcgtgttcattccattcgcgaggcctacctgccggagctgagcgttattcctggagtcaatgctgcaatatttgaggagttggaaggtcgtattttcaccgccttctctttgtatgatgctcgtaatgtcatcaagaacggtgacttcaacaatggcctgagctgctggaacgttaaagggcatgttgatgttgaagagcagaacaatcaaagatccgttctggtggtcccggagtgggaagccgaagttagccaggaggtgcgagtttgcccgggtcgtggctatattctacgtgtcaccgcatataaagagggttatggtgaaggatgtgttactatccatgagatcgaaaataataccgatgaactgaagttttcgaattgcgtggaggaagaaatctatcctaataatacagttacctgcaacgattatacggttaaccaggaagaatatggtggtgcatataccagccgcaatcgtggttataacgaagcatatgggaataacccgtcggttccagcagattatgctagcgtatatgaggaaaaatcttataccgatggtagacgtgaaaacccatgtgagttcaatcgtggttatcgtgattacacgccgctaccagtaggttatgttactaaagagctggagtactttccagaaacggacaaggtttggatcgaaattggtgaaacagaaggtacatttattgtcgacagcgtcgaactgctgctcatggaggaataa

SEQ ID NO: 19 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattttacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctaccctggaggcggagtataacctagaaagagctcaaaaagccgtgaacgcattgtttacaagcaccaaccaactgggtctgaaaacgaatgtaaccgactaccacatagatcaggtcagtaatctggtaacatatctgtccgatgagttctgcctcgatgaaaagagggaactgtctgaaaaggtcaaacatgccaaaagactgagcgatgagaggaacctccttcaggacccgaactttcggggaataaatcgccagccggaccgtggatggcgtggtagcaccgacattaccatccagggcggcgacgatgtgttcaaggaaaactacgtcaccctgagcggtacgtttgatgagtgctacccgacttacctttatcagaagatagatgaaagcaagctcaaggcatacactcgctatcagctccgcgggtacattgaggattcacaagatcttgagatttatctcatcagatacaacgcaaaacatgaaaccgttaatgtgccgggcaccggtagcctctggccgctgagcgttgaaaatcaaattggcccgtgtggcgagccgaaccggtgtgcgccacatttggagtggaatccggatctgcactgctcttgtagggacggggaaaaatgtgcccatcattcacatcactttagcctggatattgatgttgggtgtaccgatttaaatgaagatctaggtgtctgggtgatcttcaaaattaaaactcaggacggccacgcacggctgggtaacttggaatttctagaagagaaacccctggtcggagaggcgctggcccgtgttaaacgggcagaaaagaagtggcgtgacaaacgtgagaaactggaatgggagacgaacattgtgtacaaggaggcaaaagaaagcgttgatgcgctctttgtgaattctcagtacgaccagctgcaggcagatactaatattgccatgatacatgctgcagacaagcgtgttcattccattcgcgaggcctacctgccggagctgagcgttattcctggagtcaatgctgcaatatttgaggagttggaaggtcgtattttcaccgccttctctttgtatgatgctcgtaatgtcatcaagaacggtgacttcaacaatggcctgagctgctggaacgttaaagggcatgttgatgttgaagagcagaacaatcaaagatccgttctggtggtcccggagtgggaagccgaagttagccaggaggtgcgagtttgcccgggtcgtggctatattctacgtgtcaccgcatataaagagggttatggtgaaggatgtgttactatccatgagatcgaaaataataccgatgaactgaagttttcgaattgcgtggaggaagaaatctatcctaataatacagttacctgcaacgattatacggttaaccaggaagaatatggtggtgcatataccagccgcaatcgtggttataacgaagcatatgggaataacccgtcggttccagcagattatgctagcgtatatgaggaaaaatcttataccgatggtagacgtgaaaacccatgtgagttcaatcgtggttatcgtgattacacgccgctaccagtaggttatgttactaaagagctggagtactttccagaaacggacaaggtttggatcgaaattggtgaaacagaaggtacatttattgtcgacagcgtcgaactgctgctcatggaggaataa

SEQ ID NO: 20 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattatacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagccgtgaacgcattgtttacaagcaccaaccaactgggtctgaaaacgaatgtaaccgactaccacatagatcaggtcagtaatctggtaacatatctgtccgatgagttctgcctcgatgaaaagagggaactgtctgaaaaggtcaaacatgccaaaagactgagcgatgagaggaacctccttcaggacccgaactttcggggaataaatcgccagccggaccgtggatggcgtggtagcaccgacattaccatccagggcggcgacgatgtgttcaaggaaaactacgtcaccctgagcggtacgtttgatgagtgctacccgacttacctttatcagaagatagatgaaagcaagctcaaggcatacactcgctatcagctccgcgggtacattgaggattcacaagatcttgagatttatctcatcagatacaacgcaaaacatgaaaccgttaatgtgccgggcaccggtagcctctggccgctgagcgttgaaaatcaaattggcccgtgtggcgagccgaaccggtgtgcgccacatttggagtggaatccggatctgcactgctcttgtagggacggggaaaaatgtgcccatcattcacatcactttagcctggatattgatgttgggtgtaccgatttaaatgaagatctaggtgtctgggtgatcttcaaaattaaaactcaggacggccacgcacggctgggtaacttggaatttctagaagagaaacccctggtcggagaggcgctggcccgtgttaaacgggcagaaaagaagtggcgtgacaaacgtgagaaactggaatgggagacgaacattgtgtacaaggaggcaaaagaaagcgttgatgcgctctttgtgaattctcagtacgaccagctgcaggcagatactaatattgccatgatacatgctgcagacaagcgtgttcattccattcgcgaggcctacctgccggagctgagcgttattcctggagtcaatgctgcaatatttgaggagttggaaggtcgtattttcaccgccttctctttgtatgatgctcgtaatgtcatcaagaacggtgacttcaacaatggcctgagctgctggaacgttaaagggcatgttgatgttgaagagcagaacaatcaaagatccgttctggtggtcccggagtgggaagccgaagttagccaggaggtgcgagtttgcccgggtcgtggctatattctacgtgtcaccgcatataaagagggttatggtgaaggatgtgttactatccatgagatcgaaaataataccgatgaactgaagttttcgaattgcgtggaggaagaaatctatcctaataatacagttacctgcaacgattatacggttaaccaggaagaatatggtggtgcatataccagccgcaatcgtggttataacgaagcatatgggaataacccgtcggttccagcagattatgctagcgtatatgaggaaaaatcttataccgatggtagacgtgaaaacccatgtgagttcaatcgtggttatcgtgattacacgccgctaccagtaggttatgttactaaagagctggagtactttccagaaacggacaaggtttggatcgaaattggtgaaacagaaggtacatttattgtcgacagcgtcgaactgctgctcatggaggaataa

SEQ ID NO: 21 - DNA, 3498 bp, synthetic construct
  source 1..3498: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattggacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagccgtgaacgcattgtttacaagcaccaaccaactgggtctgaaaacgaatgtaaccgactaccacatagatcaggtcagtaatctggtaacatatctgtccgatgagttctgcctcgatgaaaagagggaactgtctgaaaaggtcaaacatgccaaaagactgagcgatgagaggaacctccttcaggacccgaactttcggggaataaatcgccagccggaccgtggatggcgtggtagcaccgacattaccatccagggcggcgacgatgtgttcaaggaaaactacgtcaccctgagcggtacgtttgatgagtgctacccgacttacctttatcagaagatagatgaaagcaagctcaaggcatacactcgctatcagctccgcgggtacattgaggattcacaagatcttgagatttatctcatcagatacaacgcaaaacatgaaaccgttaatgtgccgggcaccggtagcctctggccgctgagcgttgaaaatcaaattggcccgtgtggcgagccgaaccggtgtgcgccacatttggagtggaatccggatctgcactgctcttgtagggacggggaaaaatgtgcccatcattcacatcactttagcctggatattgatgttgggtgtaccgatttaaatgaagatctaggtgtctgggtgatcttcaaaattaaaactcaggacggccacgcacggctgggtaacttggaatttctagaagagaaacccctggtcggagaggcgctggcccgtgttaaacgggcagaaaagaagtggcgtgacaaacgtgagaaactggaatgggagacgaacattgtgtacaaggaggcaaaagaaagcgttgatgcgctctttgtgaattctcagtacgaccagctgcaggcagatactaatattgccatgatacatgctgcagacaagcgtgttcattccattcgcgaggcctacctgccggagctgagcgttattcctggagtcaatgctgcaatatttgaggagttggaaggtcgtattttcaccgccttctctttgtatgatgctcgtaatgtcatcaagaacggtgacttcaacaatggcctgagctgctggaacgttaaagggcatgttgatgttgaagagcagaacaatcaaagatccgttctggtggtcccggagtgggaagccgaagttagccaggaggtgcgagtttgcccgggtcgtggctatattctacgtgtcaccgcatataaagagggttatggtgaaggatgtgttactatccatgagatcgaaaataataccgatgaactgaagttttcgaattgcgtggaggaagaaatctatcctaataatacagttacctgcaacgattatacggttaaccaggaagaatatggtggtgcatataccagccgcaatcgtggttataacgaagcatatgggaataacccgtcggttccagcagattatgctagcgtatatgaggaaaaatcttataccgatggtagacgtgaaaacccatgtgagttcaatcgtggttatcgtgattacacgccgctaccagtaggttatgttactaaagagctggagtactttccagaaacggacaaggtttggatcgaaattggtgaaacagaaggtacatttattgtcgacagcgtcgaactgctgctcatggaggaataa

SEQ ID NO: 22 - DNA, 3486 bp, synthetic construct
  source 1..3486: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattttacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagctgtgaacgccctctttacctccaccaatcagcttggcttgaaaactaacgttactgactatcacattgaccaagtgtccaacttggtcacctaccttagcgatgagttctgcctcgacgagaagcgtgaactctccgagaaagttaaacacgccaagcgtctcagcgacgagaggaatctcttgcaagactccaacttcaaagacatcaacaggcagccagaacgtggttggggtggaagcaccgggatcaccatccaaggaggcgacgatgtgttcaaggagaactacgtcaccctctccggaactttcgacgagtgctaccctacctacttgtaccagaagatcgatgagtccaaactcaaagccttcaccaggtatcaacttagaggctacatcgaagacagccaagaccttgaaatctactcgatcaggtacaatgccaagcacgagaccgtgaatgtcccaggtactggttccctctggccactttctgcccaatctcccattgggaagtgtggagagcctaacagatgcgctccacaccttgagtggaatcctgacttggactgctcctgcagggatggcgagaagtgtgcccaccattctcatcacttctccttggacatcgatgtgggatgtactgacctgaatgaggacctcggagtctgggtcatcttcaagatcaagacccaagacggacacgcaagacttggcaaccttgagtttctcgaagagaaaccattggtcggtgaagctctcgctcgtgtgaagagagcagagaagaagtggagggacaaacgtgagaaactcgaatgggaaactaacatcgtttacaaggaggccaaagagtccgtggatgctttgttcgtgaactcccaatatgatcagttgcaagccgacaccaacatcgccatgatccacgccgcagacaaacgtgtgcacagcattcgtgaggcttacttgcctgagttgtccgtgatccctggtgtgaacgctgccatcttcgaggaacttgagggacgtatctttaccgcattctccttgtacgatgccagaaacgtcatcaagaacggtgacttcaacaatggcctcagctgctggaatgtgaaaggtcatgtggacgtggaggaacagaacaatcagcgttccgtcctggttgtgcctgagtgggaagctgaagtgtcccaagaggttagagtctgtccaggtagaggctacattctccgtgtgaccgcttacaaggagggatacggtgagggttgcgtgaccatccacgagatcgagaacaacaccgacgagcttaagttctccaactgcgtcgaggaagaaatctatcccaacaacaccgttacttgcaacgactacactgtgaatcaggaagagtacggaggtgcctacactagccgtaacagaggttacaacgaagctccttccgttcctgctgactatgcctccgtgtacgaggagaaatcctacacagatggcagacgtgagaacccttgcgagttcaacagaggttacagggactacacaccacttccagttggctatgttaccaaggagcttgagtactttcctgagaccgacaaagtgtggatcgagatcggtgaaaccgagggaaccttcatcgtggacagcgtggagcttctcttgatggaggaataa

SEQ ID NO: 23 - DNA, 44 bp, synthetic construct
  source 1..44: mol_type = other DNA
gagtttttctgattaacagaagcttatggaaattaacaaccaga

SEQ ID NO: 24 - DNA, 48 bp, synthetic construct
  source 1..48: mol_type = other DNA
tgccaaatgtttgaacgatcggcgcgccttattcctccatcagcagca

SEQ ID NO: 25 - DNA, 48 bp, synthetic construct
  source 1..48: mol_type = other DNA
tgccaaatgtttgaacgatcggcgcgccttattcctccatgagcagtt

SEQ ID NO: 26 - DNA, 48 bp, synthetic construct
  source 1..48: mol_type = other DNA
tgccaaatgtttgaacgatcggcgcgccttattcctccatcaagagaa

SEQ ID NO: 27 - DNA, 20 bp, synthetic construct
  source 1..20: mol_type = other DNA
cagggtgtcacgttgcaaga

SEQ ID NO: 28 - DNA, 20 bp, synthetic construct
  source 1..20: mol_type = other DNA
ccgctcgtctggctaagatc

SEQ ID NO: 29 - DNA, 24 bp, synthetic construct
  source 1..24: mol_type = other DNA
tgcctgaaaccgaactgcccgctg

SEQ ID NO: 30 - DNA, 22 bp, synthetic construct
  source 1..22: mol_type = other DNA
gagggtgttgtggctggtattg

SEQ ID NO: 31 - DNA, 23 bp, synthetic construct
  source 1..23: mol_type = other DNA
tctcaactgtccaatcgtaagcg

SEQ ID NO: 32 - DNA, 25 bp, synthetic construct
  source 1..25: mol_type = other DNA
cttacgctgggccctggaaggctag

SEQ ID NO: 33 - protein, 615 aa, synthetic construct
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYFTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKAVNALFTST

SEQ ID NO: 34 - DNA, 1848 bp, synthetic construct
  source 1..1848: mol_type = other DNA
atggaaattaacaaccagaatcaatgtgtcccgtacaactgtttaagcaatcctaaggagataattctgggtgaagagcggctagaaaccgggaacaccgttgctgatattagtctggggctgattaattttttgtattctaattttgtgccgggtggcggttttatagtaggcctgctagagcttatctggggatttatcggtccgtcccagtgggatatctttctggcccagattgagcagctgatttcacagaggattgaagaatttgctagaaaccaagcaatttccaggctcgaagggttatctaatttgtataaagtatatgtacgggcttttagcgactgggagaaagatccgaccaatccagcactgcgtgaagagatgcggattcagttcaatgacatgaacagcgcattgatcaccgcaattccgctcttccgcgtacaaaattatgaagtggcgttgctgtccgtctatgttcaagccgctaacttacacctaagcattctgcgggatgtgagtgtttttggcgaacgatggggttatgataccgcgacgatcaataatagatattccgatctgaccagcctcatccatgtttatacaaaccattgtgttgatacgtataaccagggactcagacgtctggagggtcggttcttatccgattggatcgtatataaccgttttagacgccagttgacaattagcgttctggatattgtggcgttcttcccaaattatgatattcgtacctatccgatacagactgcgactcagctgacgagagaagtttatcttgatcttccgttcatcaacgagaatctgtctcctgcagcagtttatccaaccttttctgccgctgaaagcgcaataattcgtagtccacatctggttgactttttaaattccttcacaatctataccgatagcctagcacggtcagcatactggggaggccacctggtgaatagttttcgtacgggcaccaccaccaatttgattcgttccccgttgtatggcagggaaggcaacacagaaaggccagttaccatcaccgcaagcccgagcgttcccatttttcgtactctgagctattttacgggtctggacaacagcaaccctgttgctgggattgagggtgttgagttccagaataccattagccgtagtatctacagaaaaagcggacctatcgattcctttagcgaactgcccccgcaggatgcatctgtgagcccggccattggctatagccaccgtctgtgccacgccacgtttctggagcgtatcagcgggccgcgaatcgctggtactgtgttttcctggacccatcgtagcgccagtccgaccaatgaagttagcccttctcgtatcacgcaaatcccatgggtgaaggcacataccctcgcatcgggtgcatccgtcattaagggcccgggctttaccggtggggacattctgaccagaaactccatgggtgaactcggcaccctgcgcgtaacatttactggtaggctgccccagagctactacattcgatttcgttatgccagcgtcgccaaccggagcggcacctttcgatattcccagccaccgtcctatggcatatcatttccaaaaaccatggacgcaggtgagcctctcactagccgttcgtttgcacataccaccctgttcacgccgattacgttttcccgtgcacaggaagaattcgacctctatatccagagtggcgtttacatcgaccgcatcgaatttattccggttacggctacctttgaggcggagtatgatctagaaagagctcaaaaagctgtgaacgccctctttacctccacctaa

SEQ ID NO: 35 - DNA, 45 bp, synthetic construct
  source 1..45: mol_type = other DNA
tgccaaatgtttgaacgatcggcgcgccttaggtggaggtaaaga